SEQUENCE LISTING (102 sequences)

SEQ ID NO: 1 - protein, 19 aa, Parechovirus Ljungan virus
PTTSNITWESPLGEHSWGS

SEQ ID NO: 2 - protein, 19 aa, Parechovirus Ljungan virus
PTTSNLVWESPTGMSSWGS

SEQ ID NO: 3 - protein, 16 aa, Parechovirus Ljungan virus
PTTSNLVWQGLHSWGS

SEQ ID NO: 4 - protein, 16 aa, Parechovirus Ljungan virus
PTTSNIVWEGLHSWGS

SEQ ID NO: 5 - protein, 14 aa, Parechovirus Human Parechovirus
PTGSVVTFQNSWGS

SEQ ID NO: 6 - protein, 31 aa, Parechovirus Ljungan virus
HDIEMDHSTKCFLNQCGDVEENPGPDIELVY

SEQ ID NO: 7 - protein, 31 aa, Parechovirus Ljungan virus
HDKDMDYAGGKFLNQCGDVETNPGPDIELVY

SEQ ID NO: 8 - protein, 31 aa, Parechovirus Ljungan virus
HSDEMDFAGGKFLNQCGDVETNPGPDIELVY

SEQ ID NO: 9 - protein, 31 aa, Parechovirus Ljungan virus
HNDEMDYSGGKFLNQCGDVESNPGPDIELVY

SEQ ID NO: 10 - protein, 20 aa, Parechovirus Human Parechovirus
LTNQSPYGQQPQNRMMKLAY

SEQ ID NO: 11 - protein, 24 aa, Parechovirus Ljungan virus
MVDIHPIGQEPFQNQSWRDFNEVS

SEQ ID NO: 12 - protein, 24 aa, Parechovirus Ljungan virus
MVDIHSPDQPPFQNQSWRDFNEVS

SEQ ID NO: 13 - protein, 22 aa, Parechovirus Ljungan virus
MVNFVPMEQMRQEGWREFNDVS

SEQ ID NO: 14 - protein, 24 aa, Parechovirus genus, Ljungan virus
MVDIHPIGAEPFQNQGFREFNDVS

SEQ ID NO: 15 - protein, 15 aa, Parechovirus genus, human parechovirus
ILSNQGPFKGFNEVS

SEQ ID NO: 16 - protein, 16 aa, Parechovirus Ljungan virus
KKEKKEEKEERAYNPQ

SEQ ID NO: 17 - protein, 16 aa, Parechovirus Ljungan virus
KKEKKEDKQERAYNPQ

SEQ ID NO: 18 - protein, 17 aa, Parechovirus Ljungan virus
KKEKKEEERQERAYNPQ

SEQ ID NO: 19 - protein, 14 aa, Parechovirus Human Parechovirus
KKEESKDERAYNPT

SEQ ID NO: 20 - protein, 22 aa, Parechovirus Ljungan virus
VHLKAATWLTSHNRTHEVLRIE

SEQ ID NO: 21 - protein, 22 aa, Parechovirus Ljungan virus
VHLAAAQWQTTHNRTHEVLRIE

SEQ ID NO: 22 - protein, 22 aa, Parechovirus Ljungan virus
VHLAHGTWTTNQHRQALVASIT

SEQ ID NO: 23 - protein, 22 aa, Parechovirus Ljungan virus
VHLANGTWTTSQHRQSLVASIQ

SEQ ID NO: 24 - protein, 22 aa, Parechovirus Human Parechovirus
VRLETHEWTPSWARGYQITHVE

SEQ ID NO: 25 - protein, 32 aa, Parechovirus Ljungan virus
CLIACYLPKTALDHFDSYTFGTFTNFPHVLMN

SEQ ID NO: 26 - protein, 32 aa, Parechovirus Ljungan virus
CLIACYLPATALENFNSYTFGTFTNFPHVLMN

SEQ ID NO: 27 - protein, 32 aa, Parechovirus Ljungan virus
CLIAAYMPKTAHDHMNTYTFGSYTNLPHVLMN

SEQ ID NO: 28 - protein, 32 aa, Parechovirus genus, Ljungan virus
CLIAAYMPKTAHDHMGTYTFGSYTNLPHVLMN

SEQ ID NO: 29 - protein, 32 aa, Parechovirus genus, Ljungan virus
CLIAAYMPKSAHDHMDSYTFSSYTNLPHVLMN

SEQ ID NO: 30 - protein, 32 aa, Parechovirus Human Parechovirus
SALVVYEPKPVVTYDSKLEFGAFTNLPHVLMN

SEQ ID NO: 31 - protein, 38 aa, Parechovirus genus, Ljungan virus
LMDIARMPSCLRGERATPADSQGYFQWSANTTPLNFIF

SEQ ID NO: 32 - protein, 38 aa, Parechovirus genus, Ljungan virus
LMDLARMPSCLRGERAVPQDTTGYFTWSSNITPLNFIF

SEQ ID NO: 33 - protein, 38 aa, Parechovirus Ljungan virus
LMEIARMPSVFLGESTEPDGRRGYFTWSHTISPVNWVF

SEQ ID NO: 34 - protein, 38 aa, Parechovirus Ljungan virus
LMEIARMPSVYKGERTEPGGTNGYFTWSHTHSPINWVF

SEQ ID NO: 35 - protein, 42 aa, Parechovirus Human Parechovirus
LVKIAQLFSVMADSTTPSENHGVDAKGYFKWSATTAPQSIVH

SEQ ID NO: 36 - protein, 29 aa, Parechovirus Ljungan virus
GRLRMAFFPNQHENFVEPTAQNALYVVCD

SEQ ID NO: 37 - protein, 29 aa, Parechovirus Ljungan virus
GRLRMAFFPNQGENFGEPAAQNSIYVVCD

SEQ ID NO: 38 - protein, 29 aa, Parechovirus Ljungan virus
GRLRMAFFPNREGAYTQDEAQNAIFVVCD

SEQ ID NO: 39 - protein, 29 aa, Parechovirus Ljungan virus
GRLRMAFFPNHDARYTEEEAQNAIFMVCD

SEQ ID NO: 40 - protein, 28 aa, Parechovirus Human Parechovirus
GRLRMGFFPNATTDSTSTLDNAIYTICD

SEQ ID NO: 41 - protein, 29 aa, Parechovirus Ljungan virus
AWRHTITNIDNANVGRFTIPFPTTTHGAL

SEQ ID NO: 42 - protein, 29 aa, Parechovirus Ljungan virus
AWRFQITNLDNSSIGRFTIPFPTTTHGSL

SEQ ID NO: 43 - protein, 29 aa, Parechovirus Ljungan virus
AWAVGVFNTETAAIQKFDLHFPTSTHGAL

SEQ ID NO: 44 - protein, 29 aa, Parechovirus Ljungan virus
AWAVGIFNVTNANISKFDLNFPTTTHGAL

SEQ ID NO: 45 - protein, 29 aa, Parechovirus genus, human parechovirus
AWFFMEHTFTNEGQWRVPLEFPKQGHGSL

SEQ ID NO: 46 - RNA, 587 nt, Parechovirus Ljungan virus
ggucgaagccgcuuggaauaagacuggugcuguucacaguguguucacgaucuaugcaacuucugucuuccaucugugucuaugcagguggauaagggcuguacccgggcgguccuacucuccacaggggccugcguagguggcuuucaccucuggacagcgcaccccaccccgccuacugagugaagggcauacggcuuuucuauugcgaauuguauugaaguuuaggauuagguguagcgaccacaugugugccagcggaucuccccuggugguaacaccagccucugggcccaaaaggcaugucucugaccacacaugugcacaaccccagugauaauccuuuuuaguaaaugcuuaguaacaguggauuagcagugcuccucaagcucaguagggcuuagguauggcagguugaaggaugcccuggagguacccgcagguaacgauaaguucacuguggaucugaccaggggcccaccgggaauuccccggguagaagccuucgggccuuggguuaaaaaacgucuagaccugucccccagagggaucugggguuucccuuuuaaccccaauuauucaauaug

SEQ ID NO: 47 - RNA, 38 nt, Parechovirus Ljungan virus
uuugagugauuuauuuugauugcuuuugcuuuugcuuu

SEQ ID NO: 48 - RNA, 44 nt, Parechovirus Ljungan virus
uuugagugauucuuuucuuuuugcuuugcuuuuugcuuugcuuu

SEQ ID NO: 49 - RNA, 50 nt, Parechovirus Ljungan virus
uuugauugaauugauuuaguuugauuuugauuuuauuagcuuuaguuuau

SEQ ID NO: 50 - RNA, 50 nt, Parechovirus Ljungan virus
uuugauuaaauugauuuaauuugauuuugauuuuguuaguuuuaguuuaa

SEQ ID NO: 51 - RNA, 49 nt, Parechovirus Ljungan virus
uuugauuagauugauuuaguuugauuuugauuuuauuaguuuuauuuua

SEQ ID NO: 52 - RNA, 38 nt, Parechovirus Ljungan virus
uagaauagaauugccugcauaaaaagauuuugauuuga

SEQ ID NO: 53 - RNA, 41 nt, Parechovirus Ljungan virus
uaguuuaguuuugcuuuugcuuuuaaaagauuuugauuuga

SEQ ID NO: 54 - RNA, 50 nt, Parechovirus Ljungan virus
guaaguuagaauuagauuauuuuaguuuaguuuuaaagauuuugauuuga

SEQ ID NO: 55 - RNA, 50 nt, Parechovirus Ljungan virus
guaaguuagaauuagauuauuuuaauuuagcuuuaaagauuuugauuuga

SEQ ID NO: 56 - RNA, 47 nt, Parechovirus Ljungan virus
gguuagaauuagauuauuuuaguuuaguuuuaaggauuuugauuuga

SEQ ID NO: 57 - RNA, 30 nt, Parechovirus Ljungan virus
uugaauuuggcccaccaaucaaaaaaaaaa

SEQ ID NO: 58 - RNA, 30 nt, Parechovirus Ljungan virus
uugaauuaggcccaccaaucaaaaaaaaaa

SEQ ID NO: 59 - RNA, 106 nt, Parechovirus Ljungan virus
uuugagugauuuauuuugauugcuuuugcuuuugcuuuuagaauagaauugccugcauaaaaagauuuugauuugauugaauuuggcccaccaaucaaaaaaaaaa

SEQ ID NO: 60 - RNA, 115 nt, Parechovirus Ljungan virus
uuugagugauucuuuucuuuuugcuuugcuuuuugcuuugcuuuuaguuuaguuuugcuuuugcuuuuaaaagauuuugauuugauugaauuaggcccaccaaucaaaaaaaaaa

SEQ ID NO: 61 - RNA, 130 nt, Parechovirus Ljungan virus
uuugauugaauugauuuaguuugauuuugauuuuauuagcuuuaguuuauguaaguuagaauuagauuauuuuaguuuaguuuuaaagauuuugauuugauugaauuuggcccaccaaucaaaaaaaaaa

SEQ ID NO: 62 - RNA, 47 nt, Parechovirus Ljungan virus
gggccuacaacccucaaacugcaacucccaagaagggagguaagccc

SEQ ID NO: 63 - RNA, 47 nt, Parechovirus Ljungan virus
gggccuacaacccucaaacugcaauucccaagaagggagguaggccg

SEQ ID NO: 64 - RNA, 47 nt, Parechovirus Ljungan virus
gggcuuacaacccucaaacugcaauuucuaagaagggggguaagccu

SEQ ID NO: 65 - RNA, 47 nt, Parechovirus Ljungan virus
gagcuuacaacccucaaacugcaacuuuuaagaagggggguaagccc

SEQ ID NO: 66 - RNA, 47 nt, Parechovirus Ljungan virus
gggcuuacaacccucaaacugcaacucccaagaagggggguaagcca

SEQ ID NO: 67 - protein, 16 aa, Parechovirus Ljungan virus
MAGRNETNVSELLSTV

SEQ ID NO: 68 - protein, 16 aa, Parechovirus Ljungan virus
MAASKMNPVGNLLSTV

SEQ ID NO: 69 - protein, 16 aa, Parechovirus Ljungan virus
MAATKMNPVENLLSTV

SEQ ID NO: 70 - protein, 32 aa, Parechovirus Human Parechovirus
METIKSIADMATGVVSSVDSTINAVNEKVESV

SEQ ID NO: 71 - protein, 20 aa, Parechovirus Ljungan virus
PRVPGAAVDIYSQGKRQVRK

SEQ ID NO: 72 - protein, 20 aa, Parechovirus Ljungan virus
PRVPGTAIGIFSQGRRQMRK

SEQ ID NO: 73 - protein, 20 aa, Parechovirus Ljungan virus
PRPPGADTVIYTQGKRTVRK

SEQ ID NO: 74 - protein, 20 aa, Parechovirus Ljungan virus
PRPPGAATVIYTQGKRTVRK

SEQ ID NO: 75 - protein, 20 aa, Parechovirus Ljungan virus
PRPPGANTVIFTQGKRTARK

SEQ ID NO: 76 - protein, 20 aa, Parechovirus genus, human parechovirus
PRVFAQDVNIYDNAPNGKKK

SEQ ID NO: 77 - protein, 20 aa, Parechovirus Ljungan virus
SLQMVYNQGVDGDASGLTLL

SEQ ID NO: 78 - protein, 20 aa, Parechovirus Ljungan virus
SLQIVYNQGIEGDASGLTLL

SEQ ID NO: 79 - protein, 20 aa, Parechovirus Ljungan virus
SLQTVYNQSIEGDASGLTLL

SEQ ID NO: 80 - protein, 20 aa, Parechovirus Human Parechovirus
TIKEFFNHAIDGDEQGLSLL

SEQ ID NO: 81 - protein, 27 aa, Parechovirus Ljungan virus
WKYKLTQLRNQMTPGDSIGRILEPMEE

SEQ ID NO: 82 - protein, 27 aa, Parechovirus Ljungan virus
WKYKLNQIRNEMVPGDSIGRILEPMEE

SEQ ID NO: 83 - protein, 27 aa, Parechovirus Ljungan virus
WKRKLNTIRNEMSPGSSTGRIFEPLEE

SEQ ID NO: 84 - protein, 27 aa, Parechovirus Ljungan virus
WKYKLNQIRNEMSPGDSIGRILDPMEE

SEQ ID NO: 85 - protein, 15 aa, Parechovirus Human Parechovirus
WKQQLENQTLDDLDD

SEQ ID NO: 86 - protein, 20 aa, Parechovirus Ljungan virus
LVKPTNFVNEAPYMQDLEHC

SEQ ID NO: 87 - protein, 20 aa, Parechovirus Ljungan virus
LMKSTNFVNEAPYMQDLEHC

SEQ ID NO: 88 - protein, 20 aa, Parechovirus Ljungan virus
LVKTTNFVNEAPYMQDLEHC

SEQ ID NO: 89 - protein, 20 aa, Parechovirus Ljungan virus
LVKNTNFVNEAPYMQDLEHC

SEQ ID NO: 90 - protein, 20 aa, Parechovirus Ljungan virus
LVKTTNFINEAPYMQDLEHC

SEQ ID NO: 91 - protein, 20 aa, Parechovirus Human Parechovirus
PVSQREFKNEAPYDGQLEHI

SEQ ID NO: 92 - protein, 18 aa, Parechovirus Ljungan virus
TNGNFQDQGVVVKKDKLE

SEQ ID NO: 93 - protein, 18 aa, Parechovirus Ljungan virus
TNGNMQDQGIVVKKEKLE

SEQ ID NO: 94 - protein, 20 aa, Parechovirus Ljungan virus
SNGAAGFMDQGVVVAKEKLQ

SEQ ID NO: 95 - protein, 20 aa, Parechovirus Ljungan virus
SNGASGFMDQGVVVAKEKMQ

SEQ ID NO: 96 - protein, 17 aa, Parechovirus Human Parechovirus
KNDMSDQGIVTEITPIQ

SEQ ID NO: 97 - protein, 24 aa, Parechovirus Ljungan virus
LNHWRVCMQDYEVVLHRMLRYVFE

SEQ ID NO: 98 - protein, 24 aa, Parechovirus Ljungan virus
LNQWRVCMQDYDVVLHRMLRYVFE

SEQ ID NO: 99 - protein, 24 aa, Parechovirus Ljungan virus
LNQWRVCMQDYEVALHRMLRYVFD

SEQ ID NO: 100 - protein, 24 aa, Parechovirus Human Parechovirus
LQEWNITVDDYDVVITKLMPMVFD

SEQ ID NO: 101 - DNA, 7436 bp, Parechovirus genus, Ljungan virus
ggtcgaagccgcttggaataagactggtgctgttcacagtgtgttcacgatctatgcaacttctgtcttccatctgtgtctatgcaggtggataagggctgtacccgggcggtcctactctccacaggggcctgcgtaggtggctttcacctctggacagcgcaccccaccccgcctactgagtgaagggcatacggcttttctattgcgaattgtattgaagtttaggattaggtgtagcgaccacatgtgtgccagcggatctcccctggtggtaacaccagcctctgggcccaaaaggcatgtctctgaccacacatgtgcacaaccccagtgataatcctttttagtaaatgcttagtaacagtggattagcagtgctcctcaagctcagtagggcttaggtatggcaggttgaaggatgccctggaggtacccgcaggtaacgataagttcactgtggatctgaccaggggcccaccgggaattccccgggtagaagccttcgggccttgggttaaaaaacgtctagacctgtcccccagagggatctggggtttcccttttaaccccaattattcaatatggcaggccgcaacgagactaacgtttccgagctgctttctactgtctcatctactgttggttccctgcttcaaaaccctgctgttgaggaaaaggaaatggattcggatcgtgttgctgcttccaccaccaccaatgctggaaatcttgttcaagccttggttgctcccacaatgccttttaagccggattttaagaatactgataagtttttgtgtatgagttatagccctgagactgctcctacaaatcccactaaaatggttcatcttaaagctgccacctggttgactagtcacaatcgcactcatgaggttttgaggattgaattaccacgctccttctggccagacgagaactttcccgcttggggtcagtcacgctattttggcgcagtgcgttgtggcttccattttcaggtccaattgaatgtcaacattgggtcagcgggatgcttgatcgcctgttacttacccaaaacagctttagatcattttgatagttatacttttggaacttttaccaactttccacatgtattgatgaatgctgcaacaacatctcaggcagatctttatataccctatgtttttaatcataattatgcccgcacagattcagatgatctcgggggtatctttatttatgtgtggtctgccctgacagttccaacgggagcccccaccactgtggatgttacagtctttggctccttgcttgacctggactttcaaaacccccgagtgcctggcgccgcagtggatatttattcacagggtaaaaggcaagttcggaagaccaaatccaccaaatttaaatggacccgctcaaagattgacattgctgaaggtcctggggttatgaacatctcaaatgtcctaagtacgacaggtggtcagacgactgctcttgttggtgagagggcattctatgaccccaggacagctggggccgccatccgctgcaaagatcttatggatatagcccgcatgccatcttgcttgcggggagagagagccacgcctgcagattcccagggctatttccaatggtcggccaacaccactccattgaattttatctttgaatatggtgtatattttgaggatttgcccaacttgaatctttttgctggatgctacaactattggagaggctccattgttattaaattaactgtctatgcctctactttcaacaagggccgtctgaggatggccttttttcccaaccagcatgagaactttgttgaacccactgctcaaaatgcattgtatgtggtctgtgatattggcttgaacaacacctttgaaatgacagttccctacacctggggcaactggatgcggcctacgaggggaggtcccgttggctggttgaggattgatgtacttaataggctgacctacaatagttcctccccttcaactgtcaattgcatcctgcaggtcaaaatgggcaatgatgcacgttttatggtgcccacaacatccaatatcacctgggagagcccattgggggaacattcttggggttcacagatggatttgcttgattctttggacaaccctgatgagatacaagacagtgaagaaccggagtctagcaatgttgaggcagcccaaggccaggatgcagccaaagcagttgggctgcgagccaccgagaacgatggttccctcacagaacaaataaatatggcacaaccaatgtttcttaactataaacagcataatgtggacattttttctgcatctcatactaaagtggatcatattttcggcagagcatggcgccatactattaccaatatagataatgccaatgttgggcgcttcactatcccctttcctaccacaacccatggggcattgtcacgcttctttgcctactggactggagagttgaatatacatgttttgaatatttccaacaccaatgcttttatgaaagttgcacacacctggtttggtacagattctggaattgcccgcacaggctctttggagtccaatggggtgatgataatcccaccaggtgagcaaatgacaatgtgtgttcccttttattctgaagcacctttaagaactgtaaagggcacaggtgcctctgccggccttggcgtgtttttctatcagtgtgtgggcagaagtgtgcaaaatagactagaacttttcatcagtcttcgctgtcccaatttcttttttcctgttccagccccacatgaagcctcatcaagggatgtattggcccgacttaatgatgcaactgaagaagaactggaggccgtgctgaatgctagggatccagatgagcctttgaggattggcagagcccctgaagaccctcttaaacaacttagggaggctgccaaagcctattttaaggtctaccatgacatcgagatggaccattccaccaaatgctttttgaaccagtgtggtgatgttgaagaaaaccctggccctgacatcgagctggtttacaagaaccggggattctacaagcactatgggattaggattggagatcagatttatcatcttaactctcaagatattcttaccactgccatcacaggaaaatctgagttcatcaaagagcaggatgatggaaattggacacatgccatgactgcccccttggattattttacagagaaatatgtcaattcaatggttggttctaaacatattttttcttgcaccaccaattgtgaaactattgccagggacatctttcccgggcagagtggaatttcccagtcaaaagctcttggcatagttggtctcattttactttctgcttctttactctcactcttggcagtcccctgggatctttccagcctacaaatggtgtacaaccagggtgtggatggcgatgccagtggcttgacccttttgagccaacgctgcatgatttttttttctaataccatgtgtgagacctttaataatgatttggttaagtttattattaagattttggttaggcttctttgttatatagttttgtattgtcatgcccctaatatgcttaccaccatgtgtcttggcacccttttggtcttggatattacaacctgtgaaatcttatcagcaaatactaaagccttatttcaggctcttatggatggtgatgttaagggtctagtttggaagatggctgagaatatgcaatttgcccagtcaaccgatgagcaagcggaggagatggctgccaccttttcctttgctcgtgatatggttgacatccacccaattgggcaagagcctttccaaaatcagagctggagagattttaatgaagtgagcatgtcctttcgccatgtggaatggtggttgacaatgtttaaaaagatctacaatgtcttgaagggaatttttgctcctagcatggaacagaaggctgtggcctggcttgagaaaaatcaggattatgttgctggggttcttgatcattgttcagatatgattataaagatgaaagatccaaagcagcagaggaatgcaaaggtgattgaagagtactttgaggtgctcaaaaagatgaaaccattggtgtcgctttgcattaaagttgccccctcgaccaaattttcctcacaagtctttcgcttgtattcagaactgatgaaggttaatgtgcgggtgccagctaacacagatttaacccgtatggagcccattggtatctggatttcaagtgagcctgggcaaggcaagtccttctttacccatatgctgagcacatctcttttgaaaagctgtaacttagatggagtctacaccaaccctacaggctcagaatttatggacggctatgtcggccaggatatccacatcattgatgatgctggacaaaatagagaagagaaggacctgggtttactctgccagtgcatctcatctgtgcccttcactgtacccatggctgacctgactgagaagggaaccttttacaccagcaaaattgttattgccacaactaataaaatggattttaattgcatggttttgaccgatccagctgccttggagaggcgctttcccttcaatttgaggattcgggcagttgctagctacatgacaaaggagagaaagcttgatgtgccccgttccatgggtgccatggctgacggcaactgctgggagtgctcaactgattttggtcgcacctggaagcctgtggtcatgaaagaccttgtgaaacagattacagatctttataaacagagggatgatgccttgactgtctggaaatataagttgacccagcttcggaaccagatgacacctggtgactccattggccgaatcttggaacccatggaggagaccctctgctctcttgagcgtcgctttggacagcttgcagacagtttgcgggaaaattaccatcgaaccgcagatgagttaattgaagttatagaagatctgatggctccaggccaaagtcctttttcctgcttcgaagtaactggtcctaccataaaatataccgcctgtgacaaggttaaagcctgggtgaagaaccacatgcggcggtggggagagtttgtgatgagaaatcagggctggtttactttttttactgttttgtcctcttttctttctattcttaccttggtttatttgcattataaaaaagagaaaaaggaggagaaggaggagcgggcctacaaccctcaaactgcaactcccaagaagggaggtaagcccaagctcaccctggtgaaacccacaaattttgtgaatgaagctccatacatgcaagatttggagcattgctttgcccaaacagcctatctttcatcacctgagaccaatgacatcatacactgcagtgccttgtatgaggataccatcttggtctatgggcattcacagtttttttttaatcgctatgaggacatccgcctccattttaagggcgcaatttttcctgtggatgggggtcggatttctcaggtgactgtcaatgggcaaccaatggatttgattatgttgaaaattgataaattgcccataacttttaaaaattatacaaagtattataccaatgaagtgggcagggataatctcttgatttggaattcagataaaggacgactggctatgccggttgagtgtgttacccctgccggtcctgtggaaactatggagggaacaatcacccataagacctattcctacaaggtggcttccaaacgtggtatgtgtggaggcctccttgtcaccagagtgaatggcacttttaagatcttgggaatgcatattgctggcaatggtcagatggctcgtgctgccgctgttcattttataacaaatggcaattttcaagatcagggagtggttgttaagaaggataaattagaaaaaccaatgttccttccatccaaaactgccctgagtcctagtcccctgaatggcattgtgccagtgaaaatggaaccagccgtgcttagtccccatgattctaggttggaggttgttatgcccagtgttgtgaaaagcgctgctgccaaataccgggtgaatatttttaacccggactttgagatttgggaacgcgttgtggatgatttgaaggcgcgctttcgcctgaaattgggtatacacaaacatacaactattcagaaggcgatacagggcttttcatctcttgcgtcccttgatgtttccacctctcctgggcttaagtatgtttctcaggggctcaagaagcgggatctcttgagcctagaacccttttggataagcccacagcttgaagcagatgtcaagcaggtcttggcagatgtttattcaggtaataaaccccaaactcaatttgctgctcatttgaaggatgagttgaggaagaaggagaaggttgcccaagggaaaacccgctgcattgaagcctgctctgtggattttgtggttgcctatagagttgttatgtcatcactctatgaggcaatctatcagacaccagctcaagagttaggcctggctgttgggatgaacccgtggacagattgggatccaatgataaatacacttttaccctataattatggattggattattctgcctatgatggcagcctctcggaacaacttatggcatatggcgtcgagattttagcctactgccatgaggaacccgaggtggttaaattgcttcatgccccaataattaattctcaacatgttgtgatggatgaactttggcatgtaaagggtggtatgccttctggtgccccgtgcaccactgttttgaattctatttgcaatttacttgtgtgttcctatcttgcctatgaacagagcttggatttggaagtgcgaccaattgtttatggggatgatgtaatctattctgtttcctcaccattggatatggactattttgttaagagtgctgctgaaacatttggcatggaagttactgcctctgacaaaagtggtgcaccaaaattgttgagaatggaggaaatagaatttttaaaaagaaccactaagaattttccaggctccacttataaggtgggggccttgagtctggatactatggaacaacatattatgtggatgaaaaatcttaccacattcccagagcaattggttagttttgagaatgaacttgttttgcatggaaagaatgtttatgatgattataaaaagagatttaatgaaatattgaatcattggcgtgtgtgcatgcaggattatgaagtggtcctgcaccgcatgcttcgctatgtttttgagtgatttattttgattgcttttgcttttgcttttagaatagaattgcctgcataaaaagattttgatttgattgaatttggcccaccaatc

SEQ ID NO: 102 - protein, 2254 aa, Parechovirus Ljungan virus
MAGRNETNVSELLSTVSSTVGSLLQNPAVEEKEMDSDRVAASTTTNAGNLVQALVAPTMPFKPDFKNTDKFLCMSYSPETAPTNPTKMVHLKAATWLTSHNRTHEVLRIELPRSFWPDENFPAWGQSRYFGAVRCGFHFQVQLNVNIGSAGCLIACYLPKTALDHFDSYTFGTFTNFPHVLMNAATTSQADLYIPYVFNHNYARTDSDDLGGIFIYVWSALTVPTGAPTTVDVTVFGSLLDLDFQNPRVPGAAVDIYSQGKRQVRKTKSTKFKWTRSKIDIAEGPGVMNISNVLSTTGGQTTALVGERAFYDPRTAGAAIRCKDLMDIARMPSCLRGERATPADSQGYFQWSANTTPLNFIFEYGVYFEDLPNLNLFAGCYNYWRGSIVIKLTVYASTFNKGRLRMAFFPNQHENFVEPTAQNALYVVCDIGLNNTFEMTVPYTWGNWMRPTRGGPVGWLRIDVLNRLTYNSSSPSTVNCILQVKMGNDARFMVPTTSNITWESPLGEHSWGSQMDLLDSLDNPDEIQDSEEPESSNVEAAQGQDAAKAVGLRATENDGSLTEQINMAQPMFLNYKQHNVDIFSASHTKVDHIFGRAWRHTITNIDNANVGRFTIPFPTTTHGALSRFFAYWTGELNIHVLNISNTNAFMKVAHTWFGTDSGIARTGSLESNGVMIIPPGEQMTMCVPFYSEAPLRTVKGTGASAGLGVFFYQCVGRSVQNRLELFISLRCPNFFFPVPAPHEASSRDVLARLNDATEEELEAVLNARDPDEPLRIGRAPEDPLKQLREAAKAYFKVYHDIEMDHSTKCFLNQCGDVEENPGPDIELVYKNRGFYKHYGIRIGDQIYHLNSQDILTTAITGKSEFIKEQDDGNWTHAMTAPLDYFTEKYVNSMVGSKHIFSCTTNCETIARDIFPGQSGISQSKALGIVGLILLSASLLSLLAVPWDLSSLQMVYNQGVDGDASGLTLLSQRCMIFFSNTMCETFNNDLVKFIIKILVRLLCYIVLYCHAPNMLTTMCLGTLLVLDITTCEILSANTKALFQALMDGDVKGLVWKMAENMQFAQSTDEQAEEMAATFSFARDMVDIHPIGQEPFQNQSWRDFNEVSMSFRHVEWWLTMFKKIYNVLKGIFAPSMEQKAVAWLEKNQDYVAGVLDHCSDMIIKMKDPKQQRNAKVIEEYFEVLKKMKPLVSLCIKVAPSTKFSSQVFRLYSELMKVNVRVPANTDLTRMEPIGIWISSEPGQGKSFFTHMLSTSLLKSCNLDGVYTNPTGSEFMDGYVGQDIHIIDDAGQNREEKDLGLLCQCISSVPFTVPMADLTEKGTFYTSKIVIATTNKMDFNCMVLTDPAALERRFPFNLRIRAVASYMTKERKLDVPRSMGAMADGNCWECSTDFGRTWKPVVMKDLVKQITDLYKQRDDALTVWKYKLTQLRNQMTPGDSIGRILEPMEETLCSLERRFGQLADSLRENYHRTADELIEVIEDLMAPGQSPFSCFEVTGPTIKYTACDKVKAWVKNHMRRWGEFVMRNQGWFTFFTVLSSFLSILTLVYLHYKKEKKEEKEERAYNPQTATPKKGGKPKLTLVKPTNFVNEAPYMQDLEHCFAQTAYLSSPETNDIIHCSALYEDTILVYGHSQFFFNRYEDIRLHFKGAIFPVDGGRISQVTVNGQPMDLIMLKIDKLPITFKNYTKYYTNEVGRDNLLIWNSDKGRLAMPVECVTPAGPVETMEGTITHKTYSYKVASKRGMCGGLLVTRVNGTFKILGMHIAGNGQMARAAAVHFITNGNFQDQGVVVKKDKLEKPMFLPSKTALSPSPLNGIVPVKMEPAVLSPHDSRLEVVMPSVVKSAAAKYRVNIFNPDFEIWERVVDDLKARFRLKLGIHKHTTIQKAIQGFSSLASLDVSTSPGLKYVSQGLKKRDLLSLEPFWISPQLEADVKQVLADVYSGNKPQTQFAAHLKDELRKKEKVAQGKTRCIEACSVDFVVAYRVVMSSLYEAIYQTPAQELGLAVGMNPWTDWDPMINTLLPYNYGLDYSAYDGSLSEQLMAYGVEILAYCHEEPEVVKLLHAPIINSQHVVMDELWHVKGGMPSGAPCTTVLNSICNLLVCSYLAYEQSLDLEVRPIVYGDDVIYSVSSPLDMDYFVKSAAETFGMEVTASDKSGAPKLLRMEEIEFLKRTTKNFPGSTYKVGALSLDTMEQHIMWMKNLTTFPEQLVSFENELVLHGKNVYDDYKKRFNEILNHWRVCMQDYEVVLHRMLRYVFE

(19) United States
(12) Patent Application Publication
(10) Pub. No.: US 2011/0300149 A1
(43) Pub. Date: Dec. 8, 2011

(54) LJUNGAN VIRUS

(75) Inventors: Bo Niklasson, Kalmer (SE); Conny Tolf, Kalmer (SE); Michael Lindberg, Kalmer (SE)

(73) Assignee: Apodemus AB, Kalmer (SE)

(21) Appl. No.: 13/141,758

(22) PCT Filed: Dec. 23, 2009
(86) PCT No.: PCT/GB2009/002962

§ 371 Date: Jun. 23, 2011

(30) Foreign Application Priority Data
Dec. 24, 2008 (GB) 0823560.8

(51) Int. Cl. A61K 39/125; C07H 21/02; C07K 14/085; C12Q 1/70; A61K 39/395; A61P 37/04; A61P 31/14; A61P 25/00; A61P 21/04; A61P 3/10; A61P 15/06; A61P 9/00; A61K 31/4245; C07D 413/12; C12N 7/00

(52) U.S. Cl. 424/139.1; 435/235.1; 536/23.72; 530/350; 435/5; 530/387.9; 530/389.4; 424/216.1; 424/186.1; 424/159.1; 514/364; 548/131

(57) ABSTRACT

The present invention relates to a new group of Ljungan viruses, proteins expressed by the viruses, antibodies to the viruses and proteins, vaccines against the viruses, diagnostic kits for detecting the viruses, uses of the viruses, proteins, antibodies and vaccines in therapy, uses of the viruses, proteins, antibodies and vaccines in treating diseases caused by the viruses and methods of treatment for treating diseases caused by the viruses.

### BACKGROUND OF THE INVENTION

1. Technical Field

The present invention relates to a new group of Ljungan viruses, proteins expressed by the viruses, antibodies to the viruses and proteins, vaccines against the viruses, diagnostic kits for detecting the viruses, uses of the viruses, proteins, antibodies and vaccines in therapy, uses of the viruses, proteins, antibodies and vaccines in treating diseases caused by the viruses and methods of treatment for treating diseases caused by the viruses.

2. Description of the Related Art

Ljungan virus (LV) is one of two species within the Parechovirus genus of Picornaviridae, a large family consisting of more than 300 serotypes divided into nine different genera (Stanway et al., 2005). Human parechovirus (HPeV), the second species in the Parechovirus genus, is a frequent human pathogen often isolated from children with diarrhoea and gastroenteritis (Stanway & Hyypia, 1999). Recently, several new HPeV genotypes have been characterized (Al-Sunaidi et al., 2007, Watanabe et al., 2007), and one of these genotypes, HPeV3, seem to be widely distributed as it has been isolated in Japan, Europe and North America (Abed & Boivin, 2005, Benschop et al., 2006, Boivin et al., 2005, Ito et al., 2004).

LV was first isolated from bank voles (Myodes glareolus) trapped in Medelpad and Västerbotten counties in Sweden when searching for an infectious agent causing human disease (Niklasson et al., 1998, Niklasson et al., 1999). LV has been suggested as an etiological agent of human diseases based on coinciding fluctuations of vole population in northern parts of Sweden and increasing incidences of type I diabetes mellitus, myocarditis and Guillain-Barré syndrome (Niklasson et al., 1998). Recently, LV antigens were detected by immunohistochemistry in fetal tissue samples in cases of human intrauterine fetal death (Niklasson et al., 2007).

Three LV strains were initially isolated from Swedish voles, the prototype strain, 87-012, and two genetically related strains, 174F and 145SL. Genome sequence analyses showed that the 87-012 and 174F strain were almost identical (genotype 1a), while the 145SL strain (genotype 1b) was clearly related, but genetically distinct from the two other strains (Johansson et al., 2002). Phylogenetic analyses based on the 2C protease (2C^{pro}) and 3D polymerase (3D^{pol}) sequences demonstrated that LV is closely related to HPeV, although clearly separated from this species (Johansson et al., 2002, Lindberg & Johansson, 2002) and because of these genetic differences, LV was assigned to a new species within the Parechovirus genus (Stanway et al., 2005). A fourth LV strain, M1146, isolated from montane vole (Microtus montanus) in Oregon, USA, was recently characterized (Johansson et al., 2003). The genomic sequence of M1146 is related, but distinct from all Swedish strains and constitutes therefore a second genotype within the LV species. Molecular characterization of isolated LV strains has revealed distinct features compared to other picornaviruses. Such features include a virus capsid comprising only three different structural proteins (Ekström et al., 2007a, Johansson et al., 2004, Tolf et al., 2008) and a set of two different 2A protein motifs encoded by the LV genome (Johansson et al., 2003, Johansson et al., 2002).

The inventors have now discovered and determined the genomic sequence of another virus, strain 64-7855. The 64-7855 virus was first isolated from a southern red-backed vole (Myodes gappen) trapped during arbovirus studies in the north-eastern part of USA (Whitney et al., 1970). However, at this time no characterisation of the virus was carried out and so it was not possible to classify the virus. Therefore, it was not known in which virus group the virus fell.

The inventors have found that the 64-7855 virus causes lethal central nervous disease in guinea pigs. However, the virus causes the lethal central nervous disease without any signs of encephalitis. Animal models of picornavirus infection (e.g. enterovirus and cardiovirus) normally show encephalitis (Niklasson et al., 1999) when picornavirus infection leads to lethal central nervous disease. Since the 64-7855 virus does not show encephalitis with lethal central nervous disease, this virus does not display the normal characteristics of a picornavirus.

When the inventors carried out genetic characterization of the 64-7855 strain, it revealed that the 64-7855 virus had a LV-like genome organization and phylogenetic analyses of the VP1 and 3D^{pol} protein sequences demonstrated that the 64-7855 strain was a picornavirus and, in particular, a LV. This finding was unexpected and surprising since infection with the 64-7855 virus causes lethal central nervous disease without any signs of encephalitis which is contrary to the normal finding with picornaviruses. The inventors have now determined that the 64-7855 virus constitutes a novel LV and is the fifth LV to be identified.

### SUMMARY OF THE INVENTION

The present invention is directed to this new LV and other closely related LVs. The present invention provides a Ljungan virus (LV) comprising a genomic nucleotide sequence corresponding to a sequence selected from the sequence of FIG. 7, and homologous sequences having at least 80% homology to the sequence of FIG. 7, and wherein the LV causes disease in mammals.

The sequence of FIG. 7 is a cDNA sequence derived from the RNA genome of the 64-7855 LV. Therefore, the RNA genomic sequence of the 64-7855 LV is identical to the cDNA sequence of FIG. 7 except that the base thymine (T) is replaced by the base uracil (U). In this way, the nucleotide sequence of the LV corresponds to the sequence of FIG. 7.

LV 64-7855 constitutes a new LV strain and the most closely related known LV strain (M-1146) only has a genomic sequence which is 77% homologous to the genomic sequence of LV 64-7855. Therefore, the LV defined above covers this new LV and closely related LV strains. Surprisingly, this new LV strain has been found to cause lethal central nervous disease in guinea pigs without any signs of encephalitis.

The LV of the present invention has a genomic nucleotide sequence which is at least 80% homologous to the genomic nucleotide sequence of LV 64-7855. The term “homology” as used herein refers to sequence identity. This means that at least 80% of the genomic sequence of the LV of the present invention is identical to the genomic sequence of LV 64-7855. Preferably, the LV of the present invention comprises a genomic nucleotide sequence corresponding to a sequence selected from: the sequence of FIG. 7; and homologous sequences having at least 83% homology to the sequence of FIG. 7. More preferably, the homologous sequences have at least 85% homology to the sequence of FIG. 7, more preferably still, at least 87% homology to the sequence of FIG. 7, even more preferably, at least 90% homology to the sequence of FIG. 7, more preferably still, at least 92% homology to the sequence of FIG. 7, even more preferably, at least 95% homology to the sequence of FIG. 7, more preferably still, at least 97% homology to the sequence of FIG. 7, even more preferably, at least 98% homology to the sequence of FIG. 7, more preferably still, at least 99% homology to the sequence of FIG. 7 and, most preferably, the LV of the present invention comprises a genomic nucleotide sequence corresponding to the sequence of FIG. 7.

Preferably, the LV of the present invention is isolated, i.e. it is substantially free of its natural environment. Preferably, the LV is a naturally occurring LV.

The LV can cause a number of diseases in mammals, such as rodents and humans. For example, the disease caused by the LV can be one of myocarditis, cardiomyopathia, Guillain Barre syndrome, diabetes mellitus, multiple sclerosis, chronic fatigue syndrome, myasthenia gravis, amyothrophic lateral sclerosis, dermatomyositis, polymyositis, malformation such as anencephaly and hydrocephaly, spontaneous abortion, intrauterine fetal death, lethal central nervous disease and sudden infant death syndrome.

Further LVs according to the invention can be discovered and isolated by finding a source of the virus. Typically, the source of the virus is small rodents which act as the natural reservoir or vector of the LV. The source for virus isolation/discovery can be selected/identified in different ways, such as:

1. Looking for a wild rodent such as a mouse, rat or a vole with signs and symptoms similar to the diseases linked with LV in humans (e.g. diabetes or myocarditis).

2. Screening large numbers of wild rodents by PCR using several different primer combinations targeting the conserved region of the LV genome. Preferably, these primers are specific for the LV of the invention.

3. Screening a large number of wild rodents using specific antisera. Antisera are collected from human patients with a disease linked with LV and who are living in the same geographical area as the rodents. LV infected rodents are identified by immunostaining (e. g. immunohistochemistry) of formalin fixed organs. A portion of the organ to be tested is kept without being fixed in a −70° C. freezer. The unfixed material is used for virus isolation if the immunohistochemistry gives a positive result.

Tissue for virus isolation is grinded and diluted in sterile saline or PBS. One-day old suckling mice are injected with 2-4 microlitres of the tissue suspension intracerebrally.

Generally, suckling mice that are inoculated with a virus will all die within a week of inoculation. However, LV infection displays different characteristics so that any signs or symptoms of infection in the baby mice do not develop until 10 days to 3 weeks after inoculation. The signs and symptoms are very discrete and can include slow weight increase and altered mobility. Further, only 5-10% of the animals develop symptoms. This is very unusual and would in most cases result in a negative interpretation of the isolation attempt.

Only the brain tissue from suckling mice with signs and symptoms of disease are used for passage in new one-day old suckling mice. When passed, the brains from sick suckling mice are grinded and diluted in sterile saline or PBS. One-day old suckling mice are injected with 2-4 microliters of the tissue suspension intracerebrally. Several such passages may be necessary before disease develops earlier (8-12 days) and in the majority of mice. After several passages in suckling mice LV is inoculated into tissue culture such as Vero cells for amplification and identification.

LV must be adapted to cell culture by passages of the cells. No or very discrete cytopathogenic effect is seen. The cells (not the tissue culture fluid) are passed weekly into new tissue culture bottles at a rate of 1 to 5. After 3-6 such blind passages the cells are stained using antibodies directed to the isolate. These antisera can be made by immunising adult mice with the suckling mouse brain suspension of a suspected isolate and/or by using human serum from patients with the disease caused by LV living in the same geographic region as the animals used as the source for virus isolation.

LVs can be identified serologically and/or genetically as they are related to but distinct from other members of the Picornavirus family. For example, genetically the LV genome and the polyprotein encoded thereby exhibit several exceptional features, such as the absence of a predicted maturation cleavage of VPO, a conserved sequence determinant in VP0 that is typically found in VP1 of other Picornaviruses, and a cluster of two unrelated 2A proteins. The 2A1 protein is related to the 2A protein of cardio, erbo and aphthoviruses and the 2A2 protein is related to the 2A protein of parechoviruses, kobuviruses and avian encephalomyelitis virus (Lindberg and Johansson).

LV is characterized by a chronic or long lasting infection in its rodent host and reservoir. LV can replicate and cause disease in a very broad host spectrum of animal species as well as in humans. LV infects these different species of animals as well as humans and the infection often results in a long lasting or chronic infection.

LV replicates in a wide variety of tissue culture cells giving a chronic infection with a discrete cytopathogenic effect and low viral output (in the order of 1,000-100,000 viral particles per ml supernatant).

Data generated by virus cultivation under laboratory conditions show that LV grows/replicates in a number of cell lines that originate from different tissues and different species, e. g. Vero monkey kidney; Vero E6 monkey kidney; MA-104 monkey kidney; CV-1 monkey kidney; GMK monkey kidney; A-549 human lung; Hela human cervical tissue; BHK 21 hamster kidney; RD human muscle; and L-cells mouse skin.

In living animals and humans, LV replicates in muscle tissue including heart tissue, in neural cells including the brain, in endocrine glands including the beta cells of the pancreas, the thyroid gland, and the supra renal gland.

Data have shown that LV has been found in endocrine and exocrine pancreas tissue, in endothelial cells of vessels, cells in the brain (including nerve tissue), cells of the liver, cells of the placenta and the umbilical cord, muscle tissue, heart tissue, and tissue of the thyroid gland. This data was generated by detection of virus by LV specific immunohistochemistry tests, thin section electron microscopy and by PCR in humans, bank voles, lemmings, laboratory mice, rabbits, guinea pigs, arctic foxes, and moose. This shows that LV can grow in most cell types of the body and therefore infect all organs of the body.

The present invention also provides a nucleotide sequence corresponding to the genomic nucleotide sequence of the LV described above. Therefore, the nucleotide sequence codes for a functional LV. Preferably, the nucleotide sequence is RNA. Preferably, the nucleotide sequence is isolated. This nucleotide sequence can be used, for example, as an antigenic component of a vaccine.

Further, the present invention provides a protein having an amino acid sequence which is at least 95% homologous to and has a conformation that is substantially the same as a LV protein derived from the polyprotein amino acid sequence of FIG. 8. This means that the secondary and tertiary structure of the protein will be substantially the same as the LV protein derived from the polyprotein amino acid sequence of FIG. 8. As will be appreciated by one skilled in the art, given that the protein has at least 95% homology, the conformation of the protein may not be identical to the naturally occurring protein of the 64-7855 strain. However, it should be at least very similar. Preferably, the secondary and tertiary structure is at least 60% identical to the secondary and tertiary structure of the LV protein derived from the polyprotein amino acid sequence of FIG. 8. More preferably, the secondary and tertiary structure is at least 70% identical, even more preferably, at least 80% identical, more preferably still, at least 90% identical, even more preferably, at least 95% identical and, most preferably, 100% identical. Preferably, the protein should be a functional protein. For example, if the protein is a structural protein that normally forms part of the viral coat, it should be able to interact with other structural proteins to form the viral coat. Preferably, the protein is immunogenically the same as the LV protein derived from the polyprotein amino acid sequence of FIG. 8. Therefore, the protein should induce the same immune response as the LV protein derived from the polyprotein amino acid sequence of FIG. 8. For example, the protein may have a slightly different conformation but the antigenic epitopes of the protein may be the same as the LV protein derived from the polyprotein amino acid sequence of FIG. 8. Preferably, the protein is a naturally occurring protein.

FIG. 8 shows the amino acid sequence of the polyprotein of LV 64-7855. It is from this polyprotein that the proteins of the LV are produced by cleaving the polyprotein at particular sites to release functional proteins. In this way, the LV proteins are derived from the polyprotein. This form of viral protein processing occurs in many viruses, including picornaviruses, and is well known to those skilled in the art. Therefore, a skilled person presented with a LV polyprotein sequence would readily be able to determine the sequences of the proteins contained within the polyprotein and produce such proteins. For example, such proteins include the VP0, VP3 and VP1 capsid proteins, and the viral 3C protease (3C^{pro}). FIG. 6 shows the protein cleavage sites in the polyprotein for various strains of LV. Further, Johansson et al., 2003 describes the characterisation of a LV.

The protein of the invention is homologous to a LV protein derived from the polyprotein amino acid sequence of FIG. 8. Proteins derived from the polyprotein sequence of FIG. 8 are the naturally occurring functional proteins of the 64-7855 strain.

LV 64-7855 constitutes a new LV strain and the most closely related known LV strain (M-1146) has proteins with a sequence which are between 81% and 93.4% homologous to the proteins of the 64-7855 strain. Therefore, the protein of the invention differs from the proteins of other LVs and can be specifically recognised as being from a virus which is closely related to the 64-7855 strain. For example, the protein may have epitopes which are unique to the virus which allow antibodies to specifically bind the protein of the invention but not to proteins of other known LVs. In this way, the proteins comprise epitopes which are specific to the LV of the invention.

The protein of the present invention has an amino acid sequence which is at least 95% homologous to a LV protein derived from the polyprotein amino acid sequence of FIG. 8. Preferably, the protein is at least 97% homologous a LV protein derived from the polyprotein amino acid sequence of FIG. 8, more preferably, at least 98% homologous, even more preferably, at least 99% homologous and, most preferably, identical to a LV protein derived from the polyprotein amino acid sequence of FIG. 8.

The protein of the present invention can be homologous to any LV protein. For example, the protein can correspond to any one of the VP0, VP3 and VP1 capsid proteins, and the viral 3C protease (3C^{pro}). The sequences of the LV proteins can easily be determined from the information given in FIG. 6. Preferably, the protein is homologous to a structural LV protein and, more preferably, the protein is homologous to any one of the VP0, VP1 and VP3 capsid proteins. Preferably, the protein of the present invention is isolated.

The VP0, VP1 and VP3 capsid proteins form a coat around the core of the virus. Accordingly, these proteins can be used, for example, as a vaccine to elicit an immune response that is specific to the virus so that future infection by the virus is quickly eliminated by the immune system, thus preventing disease.

The present invention also provides a virus-like particle comprising proteins which are homologous to the VP0, VP1 and VP3 capsid proteins and which are as defined above. Virus-like particles are particles which resemble the complete virus from which they are derived but lack viral nucleic acid, meaning that they are not infectious. Virus-like particles and methods for producing such particles are well known to those skilled in the art, for example as described in Jennings and Bachmann.

The present invention also provides an antibody directed against an epitope on an antigen selected from the group consisting of: the LV described above; an antigenic fragment of the LV described above; the protein described above; an antigenic fragment of the protein described above; the nucleotide sequence described above; a fragment of the nucleotide sequence described above; and the virus-like particle described above.

Preferably, the antibody is specific for the antigen so that the antibody only recognises and binds an antigen associated with the LV of the invention. In this way, the fragments of the LV, the protein and the nucleotide sequence should be unique to the LV so that the antibody binds to an epitope on the fragment but not to similar fragments from other LVs or other viruses.

The term “antibody” is well known in the art. Herein it means an immunoglobulin or any functional fragment thereof. It encompasses any polypeptide that has an antigen-binding site. It includes but is not limited to monoclonal, polyclonal, monospecific, polyspecific, non-specific, humanized, human, single-chain, chimeric, synthetic, recombinant, hybrid, mutated, grafted, and in vitro generated antibodies. The term “antibody” encompasses antibody fragments such as Fab, F (ab′) 2, Fv, scFv, Fd, dAb, and any other antibody fragments that retain antigen-binding function. Typically, such fragments would comprise an antigen-binding domain.

Furthermore, the present invention provides a composition for inducing an immune response comprising a component selected from: the LV described above; the LV described above in an attenuated form; the LV described above in a killed form; an antigenic fragment of the LV described above; the protein described above; an antigenic fragment of the protein described above; the nucleotide sequence described above; an antigenic fragment of the nucleotide sequence described above; the virus-like particle described above and a combination of two or more of the preceding components.

Preferably, the composition for inducing an immune response is a vaccine.

The term “antigenic fragment” means any fragment which can stimulate an immune response to the LV. The antigenic fragment can be any size as long as it provokes an immune response which is directed to the LV. For example, an antigenic fragment of the LV could be, for example, a fragment of RNA, a protein, a protein subunit, an oligopeptide, etc. The antigenic fragment should be unique to the LV of the invention.

Preferably the composition or vaccine further comprises an adjuvant. Suitable adjuvants are well known to those skilled in the art. The composition or vaccine can induce an immune response in a mammal so that infection of the mammal with the LV of the invention can be efficiently dealt with and eliminated without the mammal suffering from a disease associated with infection by the LV.

The present invention also provides a diagnostic kit comprising a component selected from: an antibody described above; a nucleotide probe directed against a unique portion of the genome of the LV described above; and specific primers for amplification of a portion of the genome of the LV described above.

The nucleotide probe can be any kind of probe which can bind to a unique portion of the genome of the LV to allow detection of the LV RNA. Since the nucleotide probe is directed against a unique portion of the genome of the LV, it will be specific for the LV so that it will not bind to the genome of other LVs and other viruses. The nucleotide probe can be an oligonucleotide with a complementary sequence to the sequence contained in the portion of the viral genome against which the probe is directed. For example, the oligonucleotide could be DNA, RNA, a phosphorodiamidate morpholino oligo (PMO), a 2′O-Me oligonucleotide or a locked nucleic acid (LNA). Preferably, the nucleotide probe is at least a 10 mer, more preferably, at least a 20 mer and, most preferably, at least a 30 mer. Suitable probes are well known to those skilled in the art and could easily be produced based on the sequence of the LV of FIG. 7 or the sequence of a LV isolated in the future.

Preferably, the antibody or the probe are tagged or labelled with a molecular marker to allow the antibody or the probe to be easily detected. Suitable tags or labels are well known to those skilled in the art. For example, the antibody or probe may be labelled with a radioactive isotope such as ³²P.

The primers can be any suitable primers for amplifying a portion of the genome of the LV described above using, for example, PCR. Based on the sequence in FIG. 7, which corresponds to the genomic sequence of LV 64-7855, a person skilled in the art would be able to create specific primers to allow the detection of a LV of the invention. The primers are specific for the LV of the invention, i.e. by binding to a unique portion of the genome of the LV. This allows detection of only the LV of the invention so that other viruses are not detected, thereby avoiding a false positive result. Suitable primers could easily be produced by one skilled in the art based on the sequence of the LV of FIG. 7 or the sequence of a LV isolated in the future.

This allows the diagnostic kit to be used to identify the LV of the invention, for example, LV infection in a mammal. The kit can comprise one, two or all three of the components discussed above.

In another aspect, the present invention provides a pharmaceutical composition comprising an antigen selected from: the LV described above; the LV described above in an attenuated form; the LV described above in a killed form; an antigenic fragment of the LV described above; the protein described above; an antigenic fragment of the protein described above; the nucleotide sequence described above; an antigenic fragment of the nucleotide sequence described above; the antibody described above; and the vaccine described above for use in therapy.

The present invention also provides a pharmaceutical composition comprising an antigen selected from: the LV described above; the LV described above in an attenuated form; the LV described above in a killed form; an antigenic fragment of the LV described above; the protein described above; an antigenic fragment of the protein described above; the nucleotide sequence described above; an antigenic fragment of the nucleotide sequence described above; the antibody described above; and the vaccine described above for use in the prophylactic or therapeutic treatment of a disease caused by the LV described above.

The disease caused by the LV can be any of myocarditis, cardiomyopathia, Guillain Barre syndrome, diabetes mellitus, multiple sclerosis, chronic fatigue syndrome, myasthenia gravis, amyothrophic lateral sclerosis, dermatomyositis, polymyositis, malformation such as anencephaly and hydrocephaly, spontaneous abortion, intrauterine fetal death, lethal central nervous disease and sudden infant death syndrome.

Pharmaceutical compositions according to the invention comprise the antigen described above with any pharmaceutically acceptable carrier, adjuvant or vehicle. Suitable pharmaceutically acceptable carriers, adjuvants and vehicles are well known to those skilled in the art. Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be administered orally, parenterally, by inhalation spray, topically, rectally, nasally, buccally, vaginally or via an implanted reservoir. Preferably, they are administered orally or by injection. The pharmaceutical compositions of this invention may contain any conventional non-toxic pharmaceutically-acceptable carriers, adjuvants or vehicles. The term parenteral as used herein includes subcutaneous, intracutaneous, intravenous, intramuscular, intra-articular, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the antigen is combined with emulsifying and suspending agents. If desired, certain sweetening and/or flavouring and/or colouring agents may be added.

The present invention also provides a method of prophylactic or therapeutic treatment of a disease caused by the LV described above in a mammal, the method comprising administering to the mammal a prophylactically or therapeutically effective amount of a pharmaceutical composition comprising an antigen selected from: the LV described above; the LV described above in an attenuated form; the LV described above in a killed form; an antigenic fragment of the LV described above; the protein described above; an antigenic fragment of the protein described above; the nucleotide sequence described above; an antigenic fragment of the nucleotide sequence described above; the antibody described above; and the vaccine described above.

The mammal can be any mammal which can be infected with LV and in which LV can cause disease. Preferably, the mammal is human.

The present invention also provides a method of prophylactic and/or therapeutic treatment of a mammal for a disease that is caused by infection with the LV of the invention, comprising administration to said mammal of an antivirally effective amount of an antiviral compound effective against the LV to eliminate or inhibit proliferation of said virus in said mammal and at the same time prevent and/or treat said disease in said mammal.

Preferably the mammal is selected from the group consisting of humans, horses, cattle, pigs, cats, dogs and rodents such as rats and mice.

The disease caused by LV infection may be caused by the infection of a tissue or cell type. It is known that LV is capable of growth in most cell types of the body and can therefore infect all organs of the body.

The present invention also provides an antiviral compound effective against a LV of the invention for use in the treatment of a disease in a mammal that is caused by infection of the LV of the invention.

Preferably, the antiviral compound is Pleconaril or a derivative thereof which is effective against the LV of the invention. Suitable antiviral compounds, such as Pleconaril and derivatives thereof, are set out in International Patent Application WO2004/073710.

### BRIEF DESCRIPTION OF THE DRAWINGS

The invention will now be described, by way of example only, with reference to the accompanying figures in which:

FIG. 1 shows the phylogenetic relationships of the 64-7855 strain with representative members of the nine genera of the Picornaviridae and the so far unclassified duck hepatitis virus type 1. (a) Tree based on 3D^{pol} protein sequences is rooted with two picorna-like insect viruses, the Scarbrood virus (SBV) and the infectious flacherie virus (InFV). (b) Unrooted tree based on VP1 protein of members of the Parechovirus genus. Gray spheres denote the vole species (indicated in italics) from which the LV strains have been isolated. Numbers at nodes are percentage of 500 bootstrap replicates supporting that node and bars under trees represent substitutions per site.

FIG. 2 shows the predicted polyprotein cleavage sites (a) and secondary structural features of capsid proteins (b), showing sequence variation between American and Swedish LV strains (HPeV1 is included as reference). (a) Vertical lines indicate cleavage sites between viral proteins while dark and gray columns in alignments show identical and similar residues, respectively. (b) Predicted -strands and -helices in LV and HPeV1 capsid proteins are underlined in aligned sequences and corresponding loop-regions are indicated above alignments.

FIG. 3 shows the predicted stem-loop structure of the 64-7855 5′UTR (a) compared with corresponding outlined structure of the 87-012G 5′UTR (b). Stem-loop elements are labelled according to accepted notation of picornavirus type II IRES. The shaded area of predicted 5′UTR structure of 87-012G indicates the putative 5′end sequence that remains to be determined. The first codon for initiation of translation is indicated at the 3′ end of the sequence.

FIG. 4 shows the aligned nucleotide sequences (a) and predicted secondary structures of the 3′UTR of representative members of the three LV genotypes (b). Dark background columns in alignment indicate conserved residues, while less-than and more-than (< and >) signs denote conserved nucleotides participating in predicted stem of stem-loop domain II. Two additional codons in 5′ and ten adenosine of the poly A tail were included in analyses to simulate authentic virus RNA. Stop codon of polyprotein sequences are indicated by a boxed nucleotide triplet.

FIG. 5 shows alignment (a) and predicted secondary structures (b) of a putative cre of 64-7855 and the LV prototype. (a) Dark colour indicate conserved nucleotides, while less-than and more-than (< and >) signs denote conserved nucleotides participating in predicted stem structures. (b) The AAAC sequence, which is conserved in picornavirus cre, is indicated by stars (*).

FIG. 6 shows a comparison of terminal regions and predicted cleavage sites of the polyprotein sequence of the 64-7855 strain with those of previously published LV strains (i.e., 87-012, 174F and 145SL) and the HPeV prototype strain 1 (Harris).

FIG. 7 shows a cDNA nucleotide sequence corresponding to the genomic RNA sequence of LV strain 64-7855.

FIG. 8 shows the amino acid sequence of the polyprotein of LV strain 64-7855.

### DESCRIPTION OF INVENTION

Ljungan virus (LV) was discovered twenty years ago in Swedish bank voles (Myodes glareolus, previously referred to as Clethrionomys glareolus) during search for an infectious agent causing lethal myocarditis in young athletes. Previously, the genomes of four LV isolates, including the prototype 87-012 strain, have been characterized. In addition to three Swedish LV strains isolated from bank voles, which constitute two different variants of one genotype (1a and 1b), the nucleotide sequence of an American virus (M1146), isolated from a montane vole (Microtus montanus) in western USA, demonstrated the existence of a second LV genotype.

Here, the inventors present genomic analyses of a fifth LV strain (64-7855) isolated from a southern red-backed vole (Myodes gapperi) trapped during arbovirus studies in the New York state in the north-eastern USA in the 1960s. Sequence analysis of the 64-7855 genome showed an LV-like genome organization and sequence similarity to other LV strains including conserved cleavage sites within the polyprotein. Genetic and phylogenetic analyses of evolutionary relationship between the 64-7855 strain and viruses within the Picornaviridae including previously published LV strains, demonstrated that the 64-7855 strain constitutes a novel LV strain. This strain could be considered to constitute a third genotype within the LV species.

### Methods

### Viruses and Sequences

64-7855 LV was generated by passages in infant mice by intracerebral inoculation, as previously described (Beaty et al., 1989). Briefly, a filtered 10% suspension of infected mouse brain in phosphate-buffered saline was administered into 2-3 days old outbred albino mice (ICR strain) by intracerebral inoculation. After the inoculation, the mice were observed for signs of illness; when the animals were sick or moribund (about 7-8 days), they were sacrificed and their brains collected. The nucleotide (nt) sequences (and the polyproteins derived from these sequences) of picornaviruses with their corresponding accession numbers that were used in this study are as follows: Aphthovirus genus, foot-and-mouth disease virus (FMDV) (M10975) and equine rhinitis A virus (ERAV) (L43052); Cardiovirus genus, encephalomyocarditis virus (EMCV) (M22457) and Theiler's murine encephalomyelitis virus (TMEV) (M20301); Enterovirus genus, human poliovirus strain Sabin 1 (PV1S) (V01150) and A-2 plaque virus (A2pV) (NC_{—}003988); Erbovirus genus, equine rhinitis B virus (ERBV) (X96871); Hepatovirus genus, hepatitis A virus (HAV) (M59810), avian encephalomyelitis virus (AEV) (AJ225173); Kobuvirus genus, aichi virus (AiV) (NC_{—}001918); Parechovirus genus, human parechovirus 1 strain Harris (HPeV1) (L02971), HPeV3 strain A308/99 (AB084913), HPeV6 strain BNI-67/03 (EU024629), LV strain 87-012 (AF327920), LV strain 87-012G (EF202833), LV strain 174F (AF327921), LV strain 145SL (AF327922) and LV strain M1146 (AF538689); Rhinovirus genus, human rhinovirus 2 (HRV2) (X02316); and Teschovirus genus, porcine teschovirus 1 (PTV1) (NC_{—}003985). Presently unclassified duck hepatitis virus 1 strain DRL-62 (DHV1DRL) (DQ219396) and duck hepatitis virus strain AP-03337 (DHV1AP) (DQ256132) were also included in phylogenetic analysis based on 3D^{pol} protein sequences. In addition, the sequences of two insect viruses, which are distantly related to picornaviruses, the scarbrood virus (SBV) (NC_{—}002066) and the infectious flacherie virus (InFV) (NC_{—}003781), were used as out-group in the phylogenetic analysis of the 3D^{pol} protein. Sequence homologs of LV present in the structural data base (see below), the Theiler's encephalomyelitis virus strain Bean (POLG_TMEVB) (P08544) PDB ID (1tmf) and the Cricket paralysis virus (POLG_CRPV) (P13418) PDB ID (1b35), were used as references for secondary structure predictions of LV capsid proteins.

### RNA Isolation and Reverse Transcription PCR (RT-PCR)

Total RNA from virus inoculated brain tissue was extracted using an Ultraspec II kit (Biotecx Laboratories, Inc., USA) or a RiboPure kit (Ambion) according to the manufacturer's instructions. cDNA was generated by subjecting extracted RNA to reverse transcription (RT) using an SuperScipt III RT enzyme (Invitrogen) and the primer NotdT 27 (5′-ATAAGAATGCGGCCGCT₂₇-3′) at 50° C. for 1 h before inactivating the enzyme at 70° C. In order to amplify generated cDNA by PCR, several different primers were used. These primers were derived from aligned genomic sequences of previously published LV and HPeV strains and were later on selected by a primer walking strategy. For the PCR amplifications, a PicoMaxx high fidelity PCR system (Stratagene) was used. Resulting specific and overlapping amplicons were isolated by agarose gel electrophoresis and directly sequenced or cloned into the pGEM-T Easy vector (Promega) and thereafter sequenced. Often, a nested PCR approach was required to generate sufficient amount of amplicons. The need for nested PCR probably reflects the low amount of virus in the brain tissues. In order to sequence the 64-7855 cDNA, an ABI Prism BigDye terminator cycle sequencing reaction kit (Applied Biosystems) was used according to the manufacturer's instruction. Sequences derived from T/A cloned plasmids were generated by sequencing more than two clones for each region and by sequencing each region in both directions. Sequence data was generated using a 3130 Genetic Analyzer (Applied Biosystems) and Sequencher 4.6 (Gene Codes Corporation) was used for assembly and editing of sequences.

### Bioinformatic Analyses

The nt and amino acid (aa) sequences were aligned using the Clustal W program (Thompson et al., 1994) or by pairwise alignment for sequence identity analyses (Needleman & Wunsch, 1970). In order to align protein sequences, the BLOSUM substitution matrix were used (Henikoff & Henikoff, 1992). Before phylogenetic analyses, aligned sequences were manually edited and phylogenetic information in each dataset were evaluated by likelihood mapping (Strimmer & von Haeseler, 1997). Phylogenetic relations were reconstructed using the maximum likelihood method, as implemented in the HyPhy program using the JTT model for aa substitutions and the GTR model for nt substitutions (Jones et al., 1992, Pond et al., 2005, Tavaré, 1986). The appropriate model of sequence evolution for the RNA sequences were determined by the Modeltest program, version 3.7 (Posada & Crandall, 1998). The significance of inferred phylogenetic trees were evaluated by bootstrap analyses using the PhyML 3.0 program and 500 data sets (Guindon & Gascuel, 2003). MEGA 4.0, Treeview and NJplot were used to visualize trees (Page, 1996, Perriere & Gouy, 1996, Tamura et al., 2007). The RNAstrucure 4.6 software including the Dynalign method and the RNAalifold server (http://rna.tbi.univie.ac.at/cgi-bin/RNAalifold.cgi) were used for predictions of secondary RNA structures. Predictions by RNAalifold was based on alignments including all characterized LV strains, genotype 1, 3 and 6 of HPeV and members of Aphtho-, and Cardiovirus genuses (Hofacker et al., 2002, Mathews, 2005, Mathews et al., 2004). The RnaViz2 program was used to draw predicted secondary RNA structures (De Rijk et al., 2003). Secondary structures of LV capsid proteins were predicted by using the Jpred 3, PSIPRED 2.6 and APSSP2 methods (Cuff & Barton, 2000, Jones, 1999, Raghava, 2002). These methods use neural networks or modified example-based learning to predict secondary structures, and predicted structures are based on a set of aligned sequences, either provided by the user or generated by the PSI-BLAST server. Both types of alignments were used and compared for predictions LV capsid protein structures. Following prediction, secondary proteins structures were adjusted to homologous sequences (indicated by the Jpred 3 server) of viruses for which the structure has been determined (POLG_TMEVB and POLG_CRPV, see above under caption Viruses and sequences) and to predicted secondary structures of the capsid proteins of HPeV (Ghazi et al., 1998, Stanway et al., 1994).

### Results and Discussion

### The 64-7855 Strain is a Ljungan Virus

The 64-7855 virus was isolated more than forty years ago from a southern red-backed vole (Myodes gapperi) trapped in USA (Whitney et al., 1970) although, at this stage, the virus was not classified and so it was not known to be a picornavirus, let alone a LV. Previously, two different LV genotypes have been characterized, where the Swedish 87-012, 174F (1a) and 145SL (1b) strains, isolated from bank voles (Myodes glareolus), constitute genotype 1, and North American M1146, isolated from montane vole (Microtus montanus), has been assigned to a second genotype.

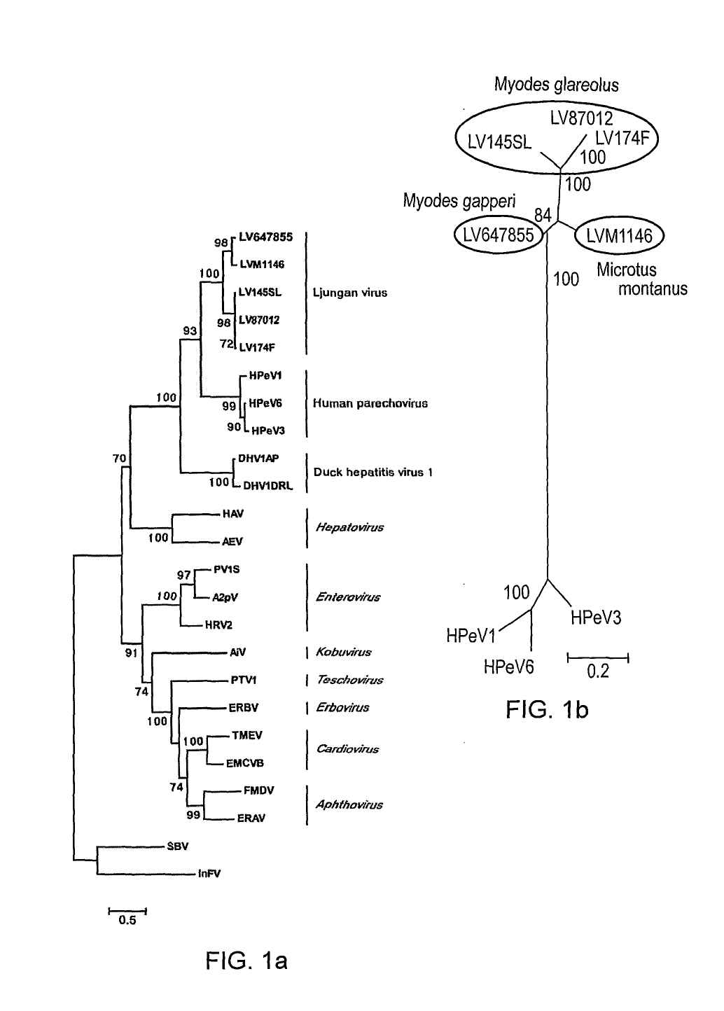

In order to position the 64-7855 strain within the family of Picornaviridae, the 3D^{pol} nucleotide sequence was first determined and compared with representative members of the different genera within the virus family. Phylogenetic relationship between the 64-7855 strain and representative members of the nine genera of Picornaviridae, demonstrated that this novel strain is related to previously characterized LV strains, and that 64-7855 and M1146 strains cluster together within the LV species (FIG. 1a).

Thereafter, the complete coding sequence, the majority of the 5′UTR and the entire 3′UTR of the 64-7855 genome were determined by sequence analyses of overlapping PCR-amplicons. Based on the VP1 proteins sequences, the phylogenetic relationships between the 64-7855 strain and previously characterized LV strains as well as members among HPeVs, verified that the 64-7855 strain is indeed a LV, and indicated, that this American isolate could represent a novel, third, genotype within the LV species (FIG. 1b).

### The 64-7855 Genome

The 64-7855 genome sequence derived from viruses propagated in mice. Analyses of the 64-7855 genome confirmed that this virus belongs to the LV species of the Picornaviridae. Molecular features associated with LV, including the presence of two different consecutive 2A protein motifs, a P1 region where the VP0 protein remains unprocessed and conserved secondary structures of the 5′ and 3′UTR, were also identified in the 64-7855 sequence. The 64-7855 genome includes a 5′UTR of 584 nt, which is most likely not completely sequenced since it is 178 nt shorter than the complete 5′UTR sequence of a previously published infectious LV clone, pLV 87-012G (Ekström et al., 2007b). Several attempts to obtain the extreme 5′-part of the 64-7855 5′UTR using different strategies including 5′RACE were unsuccessful (data not shown). Despite using conserved primer positions in stem-loop (SL) structures of 5′UTR of Swedish LV strains and HPeV, no specific amplicons were detected. Previously, a corresponding result was obtained for the American M1146 strain, suggesting that SLs in the most 5′-proximal part of the 5′UTR is not conserved between American and Swedish LV strains (Johansson et al., 2003). The 5′UTR sequence is followed by an open reading frame coding for a polyprotein of 2254 aa and the viral genome ends with a 3′UTR of 87 nt (excluding the poly A tail). The 64-7855 genome has a GC content of 45%, which is similar to the 42% of previously published LV strains and the closely related HPeV (39%) (Johansson et al., 2002). Pairwise sequence comparisons demonstrated that the majority of the 64-7855 genome share highest sequence identity with the M1146 strain, although part of the P2 and P3 regions displayed different genetic relationship (see below, Table 1). Comparative sequence analyses also showed, especially in the 5′UTR and P1 region, that the 64-7855 strain is different compared to the Swedish LV strains, but also distinct from genotype 2, exemplified by the M1146 sequence.

TABLE 1
Sequence identity between the 64-7855 strain and previously
characterized LV^{a} 87-012, 174F, 145SL and M1146 strains.

| | % Nucleotide (% amino acid) identity with 64-7855 | | | |
|---|---|---|---|---|
| Region | 87-012 | 174F | 145SL | M1146 |
| 5′- | 59.8 | 59.6 | 60.0 | 70.3 |
| UTR^{b} | | | | |
| ORF^{c} | 72.1 (79.7) | 72.9 (79.7) | 74.0 (82.4) | 77.6 (89.0) |
| P1^{d} | 67.0 (73.6) | 69.1 (73.6) | 68.8 (75.2) | 75.0 (85.9) |
| P2 | 73.3 (81.9) | 73.0 (82.0) | 77.4 (88.7) | 76.3 (88.9) |
| P3 | 76.4 (83.9) | 76.7 (83.9) | 76.6 (84.6) | 81.1 (92.2) |
| VP0 | 69.0 (78.4) | 71.9 (78.8) | 72.8 (76.8) | 78.5 (91.9) |
| VP3 | 65.7 (74.2) | 68.4 (74.2) | 68.7 (77.9) | 74.6 (85.2) |
| VP1^{e} | 66.2 (69.1) | 66.4 (68.8) | 65.2 (71.9) | 71.8 (80.6) |
| 2A2 | 73.4 (84.4) | 74.0 (84.4) | 73.7 (85.9) | 70.5 (85.2) |
| 2B | 72.9 (84.3) | 72.6 (85.0) | 81.6 (93.6) | 79.9 (91.4) |
| 2C | 73.4 (80.5) | 72.7 (80.5) | 78.4 (88.6) | 78.2 (90.4) |
| 3A | 73.2 (78.5) | 73.6 (78.5) | 74.6 (81.5) | 78.6 (89.1) |
| 3B | 85.1 (86.2) | 83.9 (89.7) | 86.2 (89.7) | 83.9 (86.2) |
| 3C | 75.5 (86.4) | 76.8 (85.4) | 77.3 (86.4) | 81.3 (92.3) |
| 3D | 76.5 (84.0) | 76.9 (84.3) | 76.2 (84.3) | 81.5 (93.4) |
| 3′-UTR | 64.5 | 62.1 | 65.2 | 78.2 |

^{a}Ljungan virus
^{b}Untranslated region
^{c}Open reading frame
^{d}Precursor region 1
^{e}VP1-encoding gene including the 2A1 motif

### The Viral Polyprotein

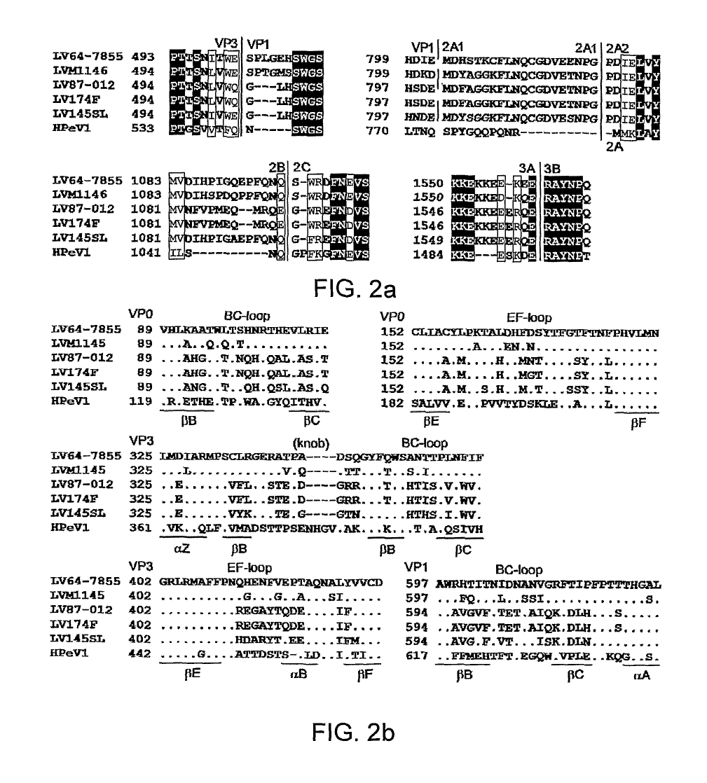

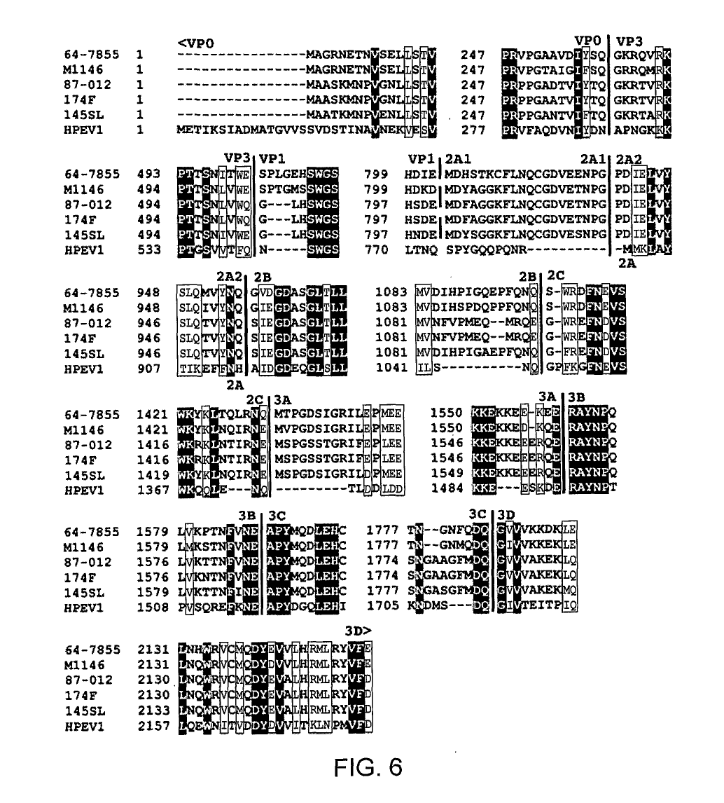

Among picornaviruses, the majority of polyprotein cleavage sites are processed by the viral 3C protease (3C^{pro}). Cleavage by 3C^{pro} is confined to restricted recognition-sites within the polyprotein (Racaniello, 2002). Primary-, secondary- and tertiary structure of these cleavage sites has been described previously (Dougherty & Semler, 1993, Palmenberg, 1990). As also been predicted for 3C^{pro} of entero-, rhino- and aphthoviruses (Blom et al., 1996), predicted cleavage sites of LV 3C^{pro} is characterized by a glutamine or glutamic acid at the P1 substrate position and a small residue at P1′ (Johansson et al., 2002). A bulky hydrophobic aa residue in the P4 position is also characteristic for LV 3C^{pro} cleavage sites. The 64-7855 polyprotein, deduced from obtained nt sequence show 89.0% identity to the American M1146 strain, while the identity to the Swedish LV strains are 79.7-82.4% (Table 1). Corresponding identity values between Swedish strains are 88.0-99.0% (data not shown). Analysis of the 64-7855 polyprotein showed that the aa sequence of the polyprotein cleavage sites are generally conserved both to previously characterized LVs and to HPeV1 (FIG. 6). There is however regions within the polyprotein where the two American LV strains, 64-7855 and M1146, demonstrate a different sequence composition than the Swedish LV strains. For instance, at the amino terminal end of the VP1 protein, an insertion of three aa residues is observed in the VP1 sequences of 64-7855 and M1146 compared to the Swedish LV strains (FIG. 2a). An insertion of two aa residues is also observed at the carboxy terminal part of 2B of the two American LV strains and 145SL, while one aa is deleted in the carboxy terminal part of 3A protein sequence of the American strains compared to the Swedish strains (FIG. 2a). The homologous positions of insertions/deletions (indels) in genomes of the American strains compared to the Swedish LV suggest a shared evolutionary history between 64-7855 and M1146 in these regions, since mechanisms resulting in indels constitute rare events in the evolution of molecular sequences.

The previously proposed processing site dividing the VP1 and 2A1 protein motifs is not conserved in the 64-7855 sequence (FIG. 2a), which support previous data indicating that the 2A1 motif is not cleaved off from the VP1 carboxy terminal end (Ekström et al., 2007a, Johansson et al., 2004, Tolf et al., 2008). Further sequence comparisons showed that main differences between the 64-7855 strain and previously characterized LV strains are found in the capsid proteins, especially in regions of predicted surface exposed loop structures. As shown in FIG. 2b, the predicted BC-loops of the VP0, VP3 and VP1 capsid proteins, the EF-loops of VP0 and VP3 and the knob region in the VP3 protein, which corresponds to an exposed region of enterovirus VP3 (Hogle et al., 1985, Muckelbauer et al., 1995), display significant sequence variation, but variations observed between the two American LV strains is less pronounced compared to the Swedish strains. For many picornaviruses, major neutralization antigenic sites are located in exposed BC- and EF-loops of the capsid proteins (Racaniello, 2002). Antisera generated against recombinant HPeV1 VP0 and VP1 proteins neutralize HPeV1 infection in cell culture (Alho et al., 2003). Antisera against recombinant VP0 and VP1 capsid proteins of the 87-012 strain detect not only viral antigens in cells infected by the prototype virus, but also viral proteins in 145SL infected cells (Tolf et al., 2008). The anti-LV antisera did not show any neutralizing effect against virus infection in cell culture by Swedish virus strains. However, observed antigenic cross-reactivity indicates that members of LV genotype 1a and 1b shares antigenic properties, and suggests furthermore, that these two variants of genotype 1 constitute one serotype. The sequence variations of predicted, exposed loop structures of the structural proteins of 64-7855 and M1146 compared to the Swedish LV strains suggests that American and Swedish LV strains would constitute different serotypes. Although no conclusive data are available, initial serological analyses supported differences between American and Swedish LV strains. Complement fixation assays using ascites fluid from LV infected animals showed a cross reactivity between 64-7855 and M1146, but no antigenic similarities to a Swedish strain, 342SL, an isolate exhibiting 99.7% sequence identity with published 145SL (data not shown).

### 5′ UTR of LV 64-7855

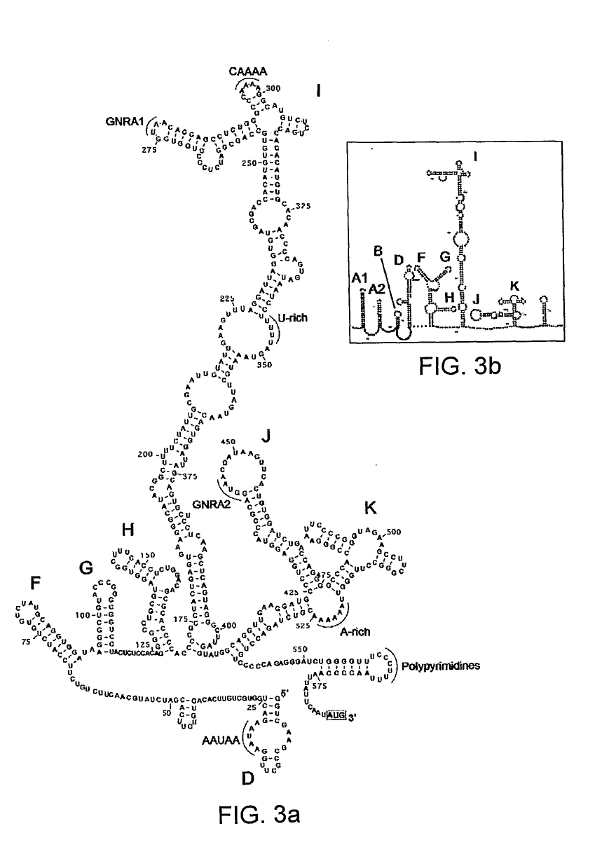

The 5′UTR secondary structure of 64-7855 was predicted using the Dynalign method (FIG. 3a) (Mathews & Turner, 2002). This is a computer algorithm that combines free energy minimization and comparative sequence analysis to find a structure that applies to two different sequences. In addition, concordant secondary structures were obtained with a thermodynamic folding minimization algorithm (MFOLD) and the RNAalifold program (data not shown). For Dynalign predictions, the sequence of previously reported 87-012G was used together with the 64-7855 sequence (FIGS. 3a and 3b). The 87-012G sequence was used since its 5′UTR sequence is completely determined and has been proven to be biologically active as it is included in an infectious cDNA clone (Ekström et al., 2007b). In the 64-7855 sequence, the initiation codon is located at position 585 in an optimal Kozak context (ANNAUGG) (Kozak, 1987). With the reservation that the most 5′ proximal part of the 5′UTR of 64-7855 is not entirely sequenced, the predicted secondary structure clearly corresponds to a type II internal ribosomal entry site (IRES), which has been described for aphtho-, cardio-, parechoviruses and previously characterized Swedish LV strains (Ghazi et al., 1998, Johansson et al., 2002, Le et al., 1993, Pilipenko et al., 1989). Covariance of nucleotide pairs in stems of predicted SL domains between different LV strains including 64-7855 supports the predicted structure. Five pairs of nt substitutions in stems of the F and H SL domains and 21 substitutions in stem of the I domain were detected (data not shown). Predicted SL structure of the 64-7855 5′UTR correspond to predicted structures for Swedish LV strains including 87-012, 174F and 145SL (Johansson et al., 2002). This conservation between different LV genotypes makes predicted structure of the type II IRES of LV more reliable. By analogy with cardio-, aphtho- and parechoviruses, parts of the predicted 5′UTR structure of 64-7855 and other LV strains are likely to be involved in IRES functions (Racaniello, 2001). The 64-7855 sequence regions predicted to make up the top of the most extended SL domain I, as well as the J and K domains, show considerable primary and secondary sequence identity to both the aphtho-, cardio-, and parechoviruses (FIG. 3a) (Clarke et al., 1987, Ghazi et al., 1998, Palmenberg & Sgro, 1997). The GNRA tetranucleotide (GNRA1) loop and following A/C-rich loop (CAAAA sequence stretch at position 295-299) of the SL I domain and the stem part of the J domain and the UUAAAAAA sequence at the root of the SLK domain are particularly well conserved among these picornaviruses. Previously, Johansson et al., (2002) reported a second GNRA sequence in the loop of the SL J domain of LV and HPeV. This GNRA2 is also present in predicted structure of the 64-7855 5′UTR and interestingly, also in corresponding position of a structure predicted for the aphthovirus 5′UTR (FIG. 3a and data not shown). The conservation of this GNRA teranucleotide in predicted structures of different picornavirus genus suggests a functional significance. The 5′-proximal part of the so far determined 5′UTR 64-7855 sequence, as a part of the truncated SL D domain, an AAUAA sequence is found at nt position 17-21 (FIG. 3a). This sequence is conserved in predicted 5′UTR structures of LV and HPEV and also in the Cardiovirus, EMCV (Ghazi et al., 1998, Palmenberg & Sgro, 1997).

Main differences between the 5′UTR structure of 64-7855 and previously published LV strain, except for the 5′-end sequence (putative SL A1-B domains indicated by gray colour in FIG. 3b) that the 64-7855 sequence is lacking, were located in loops of the F and K domains. Apart from the GNRA2 tetranucleotide, substantial sequence variation was also found in the loop of the SL J domain (FIG. 3a). The sequence variation suggests that these structures play a less important role in viral replication and translation.

### 3′UTR Structure

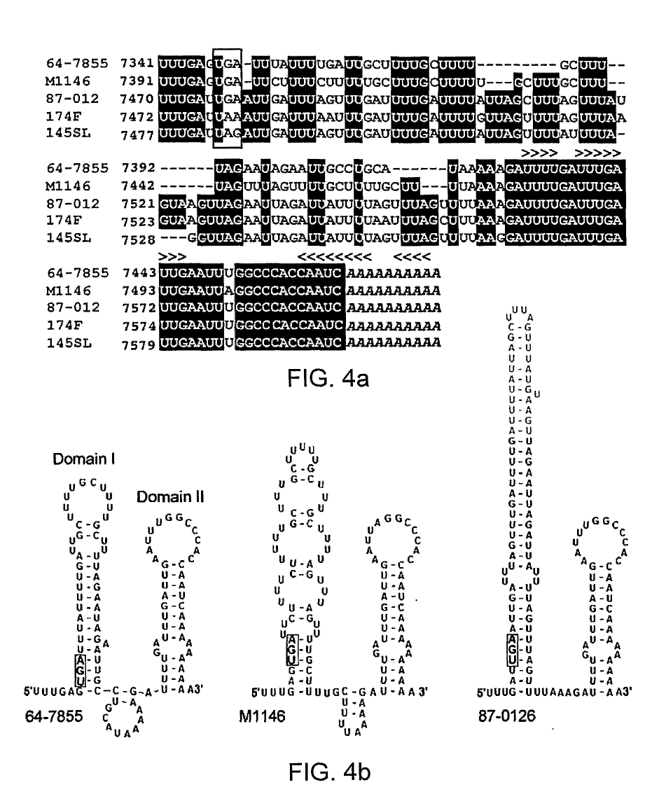

The 50-150 nt long 3′UTR of picornaviruses is important for genome replication and translation (Dobrikova et al., 2003, Rohll et al., 1995). The 3′UTR of 64-7855 is only 87 nt long compared to 96-111 nt of previously published LV strains (FIG. 4a) (Johansson et al., 2003, Johansson et al., 2002). A comparison of 3′UTRs of different LV genotypes showed that sequence differences including deletions in 64-7855, M1146 and 145SL sequences, compared to 87-012 and 174F, are found in the first of two predicted SL domains (FIGS. 4a and 4b). This part of 3′UTR is likely less important for viral replication, considering the variation of primary and predicted secondary structure of different LV strains. In contrast, the SL II domain is highly conserved among all LV genomes including the 64-7855 strain, suggesting that this structure is important for replication of the LV genome.

### A cre Sequence is Located in the LV VPg-Encoding Gene of 64-7855

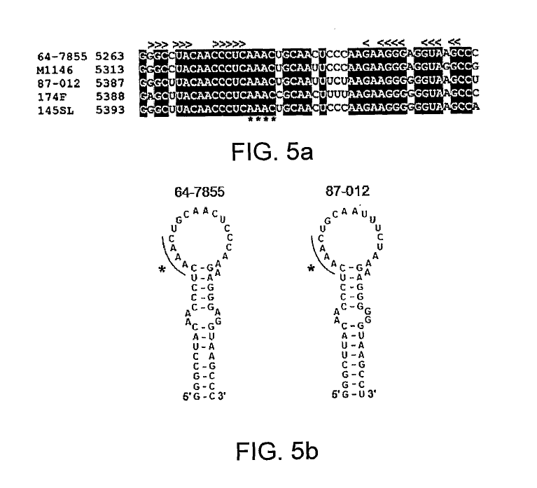

cis-acting replication elements (cre) have been recognized in genomes of several picornaviruses. These elements form short SL structures including an internal or terminal loop with three unpaired adenine (A) nucleotides. cre structures have been located in different parts of the picornavirus genome, including the 2C-encoding region of poliovirus (Goodfellow et al., 2000), the capsid-encoding regions of human rhinovirus type 14 and cardioviruses (Lobert et al., 1999, McKnight & Lemon, 1998). In addition, the existence of a cre in the VP0-encoding gene of HPeVs was recently suggested (Al-Sunaidi et al., 2007). The inventors have also suggested the location of a cre sequence in the VPg-encoding gene of the LV genome. Sequence analyses of LV genomes representing the three genotypes showed that three different regions contain conserved AAA sequences, one in the VPg gene, a second in 3C^{pro} and a third in the sequence encoding 3D^{pol} (data not shown). However, when secondary structures were predicted for these regions, only the putative sequence of the VPg gene formed a completely conserved structure around the cre sequences present in all genomes (FIGS. 5a and 5b). These results imply that the VPg gene not only codes for a protein that interacts with the cre sequence, but also contains the sequence that VPg interact with. It is tantalizing to speculate on whether this observed association has mechanistic similarities to how gene products of cellular genes may regulate their own expression.

### Activity of LV 64-7855

Five week old guinea pigs were infected with 1000 TCID50 of the virus subcutaneous. Approximately six weeks after infection the guinea pigs developed progressive general motor neuron paralysis affecting the muscles. Animals died or had to be put down within 7-14 days after debut of symptoms. Microscopic examination of the brain tissue (routine H&E staining) showed no inflammation or any other pathology.

| | | No of animals |
|---|---|---|
| Experiment | No of animals | developing disease |
| 1 | 20 | 4 |
| 2 | 20 | 7 |
| 3 | 20 | 5 |

Surprisingly, the virus caused paralysis but did not appear to cause encephalitis in contrast to other LVs.

### Concluding Remarks

Genetic and phylogenetic analyses of the 64-7855 genome clearly demonstrate that this virus belongs to the LV species of the Parechovirus genus within the family of Picornaviridae. Sequence analyses demonstrated that the 64-7855 strain could be considered to be the first member of a novel, genotype within the LV species. Specific genome features characteristic for LV, including two different 2A motifs were also found in the 64-7855 genome.

### REFERENCES

Abed, Y. & Boivin, G. (2005) J Med Virol 77, 566-570.
Al-Sunaidi et al., (2007) J Virol 81, 1013-1021.
Alho, A. et al., (2003) J Clin Microbiol 41, 2294-2299.
Beaty, B. J. et al., (1989) Arbovirues, pp. 797-855. Edited by N. J. Schmidt & R. W. Emmons. Washington D.C.: American Public Health Associations.
Benschop, K. S. et al., (2006) Emerg Infect Dis 12, 1572-1575.
Blom, N. et al., (1996) Protein Sci 5, 2203-2216.
Boivin, G. et al., (2005) Emerg Infect Dis 11, 103-105.
Clarke, B. E. et al., (1987) Nucleic Acids Res 15, 7067-79.
Cuff, J. A. & Barton, G. J. (2000) Proteins 40, 502-511.
De Rijk, P. et al., (2003) Bioinformatics 19, 299-300.
Dobrikova, E. Y. et al., (2003) Virology 311, 241-253.
Dougherty, W. G. & Semler, B. L. (1993) Microbiol Rev 57, 781-822.
Ekström, J. O. et al., (2007a) Microbiol Immunol 51, 841-850.
Ekström, J. O. et al., (2007b) Virus Res 130, 129-139.
Ghazi, F. et al., (1998) J Gen Virol 79, 2641-2650.
Goodfellow, I. et al., (2000) J Virol 74, 4590-4600.
Guindon, S. & Gascuel, O. (2003) Syst Biol 52, 696-704.
Henikoff, S. & Henikoff, J. G. (1992) Proc Natl Acad Sci USA 89, 10915-10919.
Hofacker, I. L. et al., (2002) J Mol Biol 319, 1059-1066.
Hogle, J. M. et al., (1985) Science 229, 1358-1365.
Ito, M. et al., (2004) J Gen Virol 85, 391-398.
Jennings G. T. and Bachmann M. F. (2008) Biol Chem. 389(5):521-36.
Johansson, E. S. et al., (2004) Biochem Biophys Res Commun 317, 1023-1029.
Johansson, E. S. et al., (2003) J Gen Virol 84, 837-844.
Johansson, S. et al., (2002) J Virol 76, 8920-8930.
Jones, D. T. (1999) J Mol Biol 292, 195-202.
Jones, D. T. et al., (1992) Comput Appl Biosci 8, 275-282.
Kozak, M. (1987) J Mol Biol 196, 947-950.
Le, S. Y. et al., (1993) Nucleic Acids Res 21, 2445-2451.
Lindberg, A. M. & Johansson, S. (2002) Virus Res 85, 61-70.
Lobert, P. E. et al., (1999) Proc Natl Acad Sci USA 96, 11560-11565.
Mathews, D. H. (2005) Bioinformatics 21, 2246-2253.
Mathews, D. H. et al., (2004) Proc Natl Acad Sci USA 101, 7287-7292.
Mathews, D. H. & Turner, D. H. (2002) J Mol Biol 317, 191-203.
McKnight, K. L. & Lemon, S. M. (1998) RNA 4, 1569-1584.
Muckelbauer, J. K. et al., (1995) Structure 3, 653-667.
Needleman, S. B. & Wunsch, C. D. (1970) J Mol Biol 48, 443-453.
Niklasson, B. et al., (1998) Emerg Infect Dis 4, 187-193.
Niklasson, B. et al., (1999) Virology 255, 86-93.
Niklasson, B. et al., (2007) Birth Defects Res A Clin Mol Teratol 79, 488-493.
Page, R. D. (1996) Comput Appl Biosci 12, 357-358.
Palmenberg, a. C. (1990) Annu Rev Microbiol 44, 603-623.
Palmenberg, A. C. & Sgro, J. Y. (1997) Semin virol 8, 231-241.
Perriere, G. & Gouy, M. (1996) Biochimie 78, 364-369.
Pilipenko, E. V. et al., (1989) Nucleic Acids Res 17, 5701-11.
Pond, S. L. et al., (2005) Bioinformatics 21, 676-679.
Posada, D. & Crandall, K. A. (1998) Bioinformatics 14, 817-8.
Racaniello, V. R. (2001) In Fields virology, 4 edn, pp. 658-722. Edited by D. M. Knipe, P. M. Howley, D. E. Griffin, R. A. Lamb, M. A. Matrin, B. Riozman & S. E. Straus. Philadelphia: Lippincott Williams & Wilkins.
Raghava, G. P. S. (2002). APSSP2: A combination method for protein secondary structure prediction based on neural network and example based learning. CASP5, A-132.
Rohll, J. B. et al., (1995) J Virol 69, 7835-7844.
Stanway, G. et al., (2005). Family Picornaviridae. In Virus Taxonomy. Eighth Report of the International Committee on Taxonomy of Viruses, pp. 757-778. Edited by C. M. Fauquet, M. A. Mayo, J. Maniloff, U. Desselberger & L. A. Ball. London: Elsevier/Academic Press.
Stanway, G. & Hyypiä, T. (1999) J Virol 73, 5249-5254.
Stanway, G. et al., (1994) J Virol 68, 8232-8238.
Strimmer, K. & von Haeseler, A. (1997) Proc Natl Acad Sci USA 94, 6815-6819.
Tamura, K., Dudley, J. et al., (2007) Mol Biol Evol 24, 1596-1599.
Tavaré, S. (1986). Some probabilistic and statistical problems in the analysis of DNA sequences. In Some mathematical questions in biology—DNA sequence analysis, pp. 57-86. Edited by R. M. Miura. Providence: American mathematical society.
Thompson, J. D. et al., (1994) Nucleic Acids Res 22, 4673-4680.
Tolf, C. et al., (2008) J Virol Method 150, 34-40.
Watanabe, K. et al., (2007) Emerg Infect Dis 13, 889-895.
Whitney, E. et al., (1970) J Wildl Dis 6, 48-55.

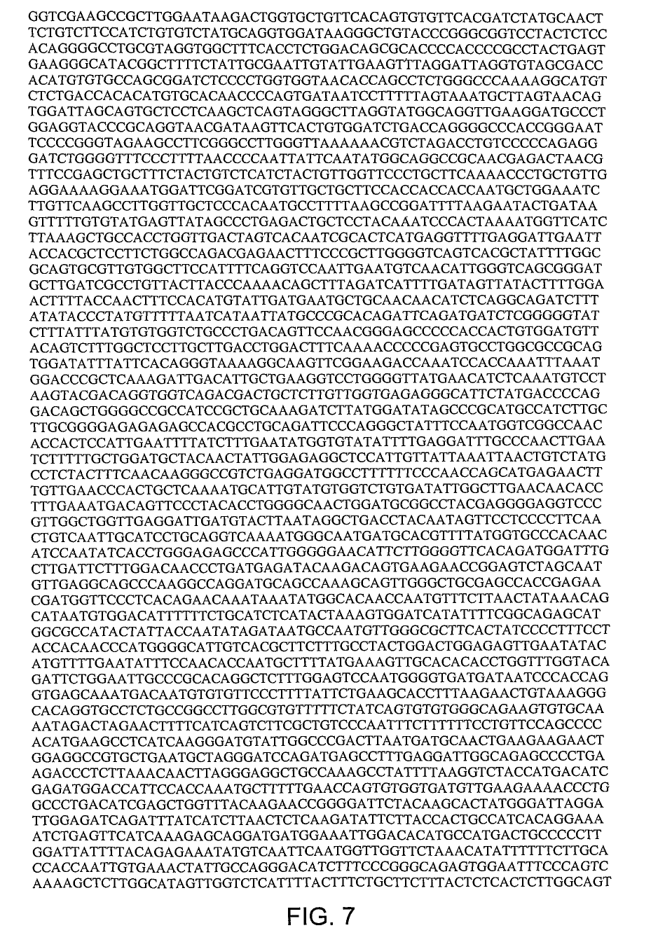

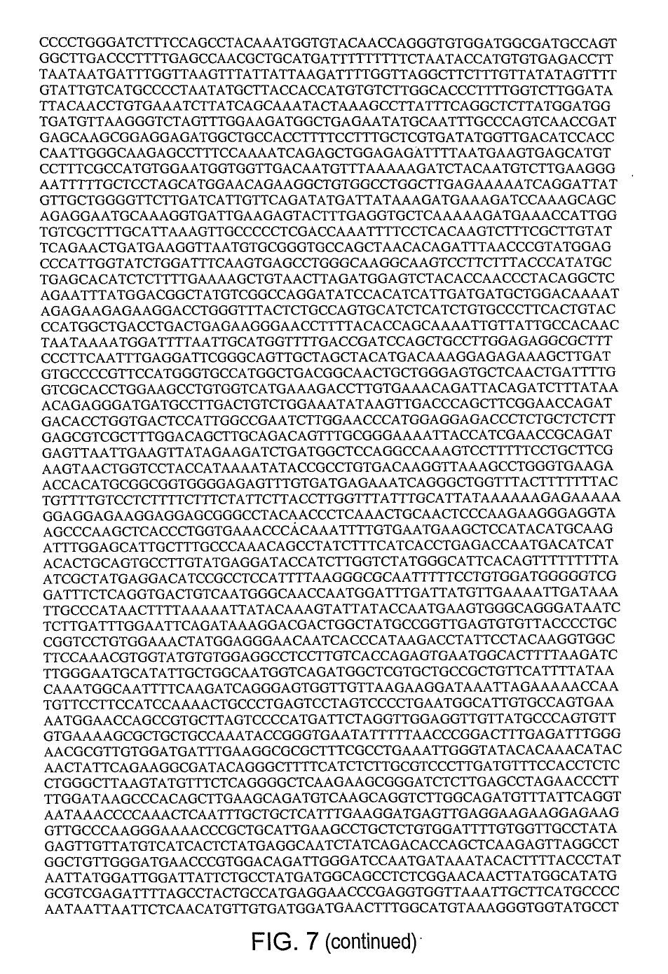

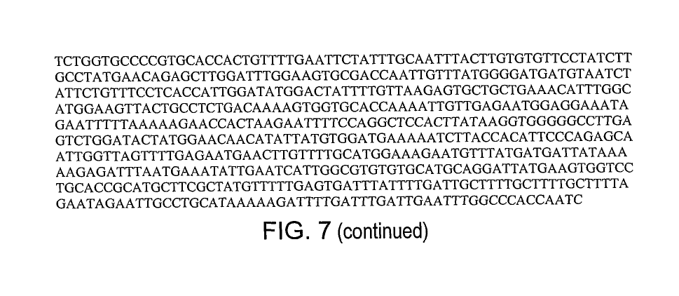

1. A Ljungan virus comprising a genomic nucleotide sequence corresponding to a sequence selected from:
the sequence of FIG. 7; and
homologous sequences having at least 80% homology to the sequence of FIG. 7,
and wherein the Ljungan virus causes disease in mammals.

2. The Ljungan virus of claim 1, wherein the homologous sequences have at least 83% homology to the sequence of FIG. 7, more preferably, at least 85% homology, even more preferably, at least 90% homology, more preferably still, at least 95% homology and, even more preferably, 98% homology.

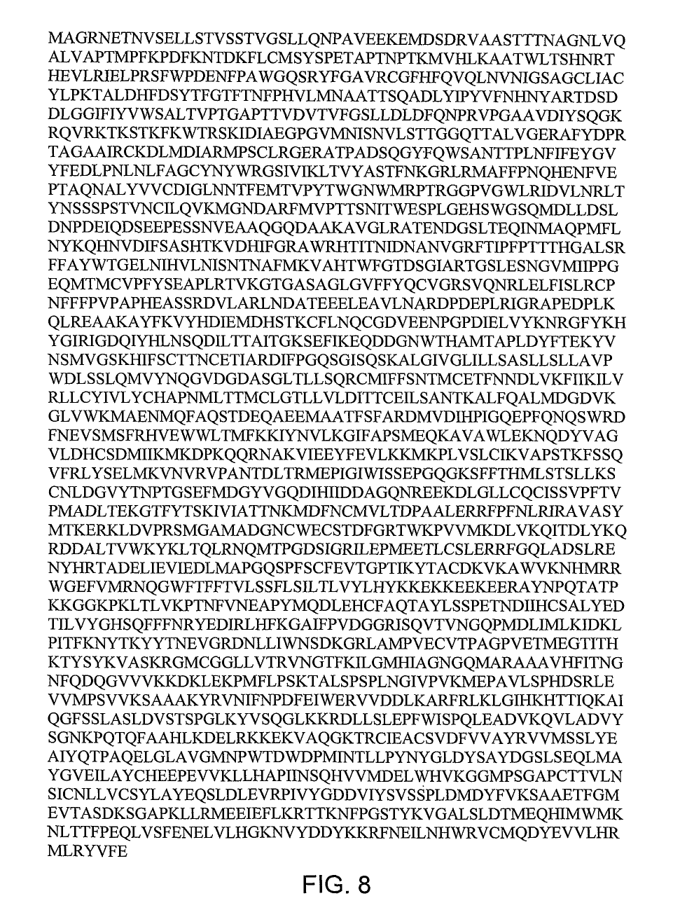

3. A protein having an amino acid sequence which is at least 95% homologous to and having a conformation that is substantially the same as a Ljungan virus protein derived from the polyprotein amino acid sequence of FIG. 8.

4. The protein of claim 3 having an amino acid sequence which is at least 97% homologous to a Ljungan virus protein derived from the polyprotein amino acid sequence of FIG. 8, more preferably, at least 98% homologous, even more preferably, at least 99% homologous to a Ljungan virus protein derived from the polyprotein amino acid sequence of FIG. 8.

5. The protein of claim 3, wherein the protein is homologous to a structural Ljungan virus protein.

6. The protein of claim 3, wherein the protein is homologous to any one of the VP0, VP1 and VP3 capsid proteins.

7. A virus-like particle comprising proteins which are homologous to the VP0, VP1 and VP3 capsid proteins and which are as defined in claim 6.

8. A nucleotide sequence corresponding to the genomic nucleotide sequence of the Ljungan virus of claim 1.

9. An antibody directed against an epitope on an antigen selected from the group consisting of: a Ljungan virus comprising a genomic nucleotide sequence corresponding to a sequence selected from: the sequence of FIG. 7; and homologous sequences having at least 80% homology to the sequence of FIG. 7, and wherein the Ljungan virus causes disease in mammals; antigenic fragment of said Ljungan virus; a protein having an amino acid sequence which is at least 95% homologous to and having a conformation that is substantially the same as a Ljungan virus protein derived from the polyprotein amino acid sequence of FIG. 8; an antigenic fragment of said protein; a virus-like particle comprising proteins which are homologous to the VP0, VP1 and VP3 capsid proteins and which are as defined in claim 6; a nucleotide sequence of corresponding to the genomic nucleotide sequence of the Ljungan virus of claim 1; and an antigenic fragment of said nucleotide sequence of claim 8.

10. The antibody of claim 8, wherein the antibody is specific for the Ljungan virus from which the antigen is derived.

11. A vaccine comprising a component selected from: a virus comprising a genomic nucleotide sequence corresponding to a sequence selected from: the sequence of FIG. 7; and homologous sequences having at least 80% homology to the sequence of FIG. 7, and wherein the Ljungan virus causes disease in mammals; said virus in an attenuated form; said virus in a killed form; an antigenic fragment of said virus; a protein having an amino acid sequence which is at least 95% homologous to and having a conformation that is substantially the same as a Ljungan virus protein derived from the polyprotein amino acid sequence of FIG. 8; an antigenic fragment of said protein; a virus-like particle comprising proteins which are homologous to the VP0, VP1 and VP3 capsid proteins and homologous to any one of the VPO, VP1, and VP3 capsid proteins; a nucleotide sequence corresponding to the genomic nucleotide sequence of the Ljungan virus of claim 1; an antigenic fragment of said nucleotide sequence of; and a combination of two or more of the preceding components.

12. A diagnostic kit comprising a component selected from: an antibody corresponding to the genomic nucleotide sequence of the Ljungan virus of claim 1; a nucleotide probe directed against a unique portion of the genome of a virus comprising a genomic nucleotide sequence corresponding to a sequence selected from: the sequence of FIG. 7; and homologous sequences having at least 80% homology to the sequence of FIG. 7, and wherein the Ljungan virus causes disease in mammals; and specific primers for amplification of a portion of the genome of said virus.

13. A pharmaceutical composition comprising an antigen selected from: a virus comprising a genomic nucleotide sequence corresponding to a sequence selected from: the sequence of FIG. 7; and homologous sequences having at least 80% homology to the sequence of FIG. 7, and wherein the Ljungan virus causes disease in mammals; said virus in an attenuated form; said virus of in a killed form; an antigenic fragment of said virus; a protein having an amino acid sequence which is at least 95% homologous to and having a conformation that is substantially the same as a Ljungan virus protein derived from the polyprotein amino acid sequence of FIG. 8; an antigenic fragment of said protein; a virus-like particle comprising proteins which are homologous to the VP0, VP1 and VP3 capsid proteins and which are as defined in claim 6; a nucleotide sequence corresponding to the genomic nucleotide sequence of the Ljungan virus of claim 1; an antigenic fragment of said nucleotide sequence; an antibody directed against an epitope on an antigen selected from the group consisting of: a Ljungan virus comprising a genomic nucleotide sequence corresponding to a sequence selected from: the sequence of FIG. 7; and homologous sequences having at least 80% homology to the sequence of FIG. 7, and wherein the Ljungan virus causes disease in mammals; antigenic fragment of said Ljungan virus; a protein having an amino acid sequence which is at least 95% homologous to and having a conformation that is substantially the same as a Ljungan virus protein derived from the polyprotein amino acid sequence of FIG. 8; an antigenic fragment of said protein; a virus-like particle comprising proteins which are homologous to the VP0, VP1 and VP3 capsid proteins and which are as defined in claim 6; a nucleotide sequence corresponding to the genomic nucleotide sequence of the Ljungan virus of claim 1; and an antigenic fragment of said nucleotide sequence; and a vaccine comprising a component selected from: a virus comprising a genomic nucleotide sequence corresponding to a sequence selected from: the sequence of FIG. 7; and homologous sequences having at least 80% homology to the sequence of FIG. 7, and wherein the Ljungan virus causes disease in mammals; said virus in an attenuated form; said virus in a killed form; an antigenic fragment of said virus; a protein having an amino acid sequence which is at least 95% homologous to and having a conformation that is substantially the same as a Ljungan virus protein derived from the polyprotein amino acid sequence of FIG. 8; an antigenic fragment of said protein; a virus-like particle comprising proteins which are homologous to the VP0, VP1 and VP3 capsid proteins and homologous to any one of the VPO, VP1, and VP3 capsid proteins; a nucleotide sequence corresponding to the genomic nucleotide sequence of the Ljungan virus of claim 1; an antigenic fragment of said nucleotide sequence; and a combination of two or more of the preceding components for use in therapy.

14. A pharmaceutical composition comprising an antigen selected from a virus comprising a genomic nucleotide sequence corresponding to a sequence selected from: the sequence of FIG. 7; and homologous sequences having at least 80% homology to the sequence of FIG. 7, and wherein the Ljungan virus causes disease in mammals; said virus in an attenuated form; said virus in a killed form; an antigenic fragment of said virus; a protein having an amino acid sequence which is at least 95% homologous to and having a conformation that is substantially the same as a Ljungan virus protein derived from the polyprotein amino acid sequence of FIG. 8; an antigenic fragment of said protein; a virus-like particle comprising proteins which are homologous to the VP0, VP1 and VP3 capsid proteins and which are as defined in claim 6; a nucleotide sequence corresponding to the genomic nucleotide sequence of the Ljungan virus of claim 1; an antigenic fragment of said nucleotide sequence; an antibody directed against an epitope on an antigen selected from the group consisting of: a Ljungan virus comprising a genomic nucleotide sequence corresponding to a sequence selected from: the sequence of FIG. 7; and homologous sequences having at least 80% homology to the sequence of FIG. 7, and wherein the Ljungan virus causes disease in mammals; antigenic fragment of said Ljungan virus; a protein having an amino acid sequence which is at least 95% homologous to and having a conformation that is substantially the same as a Ljungan virus protein derived from the polyprotein amino acid sequence of FIG. 8; an antigenic fragment of said protein; a virus-like particle comprising proteins which are homologous to the VP0, VP1 and VP3 capsid proteins and homologous to any one of the VPO, VP1, and VP3 capsid proteins; a nucleotide sequence corresponding to the genomic nucleotide sequence of the Ljungan virus of claim 1; and an antigenic fragment of said nucleotide sequence; and a vaccine comprising a component selected from: a virus comprising a genomic nucleotide sequence corresponding to a sequence selected from: the sequence of FIG. 7; and homologous sequences having at least 80% homology to the sequence of FIG. 7, and wherein the Ljungan virus causes disease in mammals; said virus in an attenuated form; said virus in a killed form; an antigenic fragment of said virus; a protein having an amino acid sequence which is at least 95% homologous to and having a conformation that is substantially the same as a Ljungan virus protein derived from the polyprotein amino acid sequence of FIG. 8; an antigenic fragment of said protein; a virus-like particle comprising proteins which are homologous to the VP0, VP1 and VP3 capsid proteins and homologous to any one of the VPO, VP1, and VP3 capsid proteins; a nucleotide sequence corresponding to the genomic nucleotide sequence of the Ljungan virus of claim 1; an antigenic fragment of said nucleotide sequence; and a combination of two or more of the preceding components for use in therapy for use in the prophylactic or therapeutic treatment of a disease caused by Ljungan virus.

15. The pharmaceutical composition of claim 14, wherein the disease caused by the Ljungan virus is selected from myocarditis, cardiomyopathia, Guillain Barre syndrome, diabetes mellitus, multiple sclerosis, chronic fatigue syndrome, myasthenia gravis, amyothrophic lateral sclerosis, dermatomyositis, polymyositis, malformation such as anencephaly and hydrocephaly, spontaneous abortion, intrauterine fetal death, lethal central nervous disease and sudden infant death syndrome.

16. A method of prophylactic or therapeutic treatment of a disease caused by a Ljungan virus comprising a genomic nucleotide sequence corresponding to a sequence selected from: the sequence of FIG. 7; and homologous sequences having at least 80% homology to the sequence of FIG. 7, and wherein the Ljungan virus causes disease in mammals in a mammal, the method comprising administering to the mammal a prophylactically or therapeutically effective amount of a pharmaceutical composition comprising an antigen selected from a virus comprising a genomic nucleotide sequence corresponding to a sequence selected from: the sequence of FIG. 7; and homologous sequences having at least 80% homology to the sequence of FIG. 7, and wherein the Ljungan virus causes disease in mammals; said virus in an attenuated form; said virus in a killed form; an antigenic fragment of said virus; a protein having an amino acid sequence which is at least 95% homologous to and having a conformation that is substantially the same as a Ljungan virus protein derived from the polyprotein amino acid sequence of FIG. 8; an antigenic fragment of said protein; a virus-like particle comprising proteins which are homologous to the VP0, VP1 and VP3 capsid proteins and homologous to any one of the VPO, VP1, and VP3 capsid proteins; a nucleotide sequence corresponding to the genomic nucleotide sequence of the Ljungan virus of claim 1; an antigenic fragment of said nucleotide sequence; an antibody directed against an epitope on an antigen selected from the group consisting of: a Ljungan virus comprising a genomic nucleotide sequence corresponding to a sequence selected from: the sequence of FIG. 7; and homologous sequences having at least 80% homology to the sequence of FIG. 7, and wherein the Ljungan virus causes disease in mammals; antigenic fragment of said Ljungan virus; a protein having an amino acid sequence which is at least 95% homologous to and having a conformation that is substantially the same as a Ljungan virus protein derived from the polyprotein amino acid sequence of FIG. 8; an antigenic fragment of said protein; a virus-like particle comprising proteins which are homologous to the VP0, VP1 and VP3 capsid proteins and homologous to any one of the VPO, VP1, and VP3 capsid proteins; a nucleotide sequence corresponding to the genomic nucleotide sequence of the Ljungan virus of claim 1; and an antigenic fragment of said nucleotide sequence; and a vaccine comprising a component selected from: a virus comprising a genomic nucleotide sequence corresponding to a sequence selected from: the sequence of FIG. 7; and homologous sequences having at least 80% homology to the sequence of FIG. 7, and wherein the Ljungan virus causes disease in mammals; said virus in an attenuated form; said virus in a killed form; an antigenic fragment of said virus; a protein having an amino acid sequence which is at least 95% homologous to and having a conformation that is substantially the same as a Ljungan virus protein derived from the polyprotein amino acid sequence of FIG. 8; an antigenic fragment of said protein; a virus-like particle comprising proteins which are homologous to the VP0, VP1 and VP3 capsid proteins and homologous to any one of the VPO, VP1, and VP3 capsid proteins; a nucleotide sequence corresponding to the genomic nucleotide sequence of the Ljungan virus of claim 1; an antigenic fragment of said nucleotide sequence; and a combination of two or more of the preceding components for use in therapy.

17. A method of prophylactic and/or therapeutic treatment of a mammal for a disease that is caused by infection with the Ljungan virus of claim 1, comprising administration to said mammal of an antivirally effective amount of an antiviral compound effective against the Ljungan virus to eliminate or inhibit proliferation of said virus in said mammal and at the same time prevent and/or treat said disease in said mammal.

18. The method of claim 17, wherein the antiviral compound is Pleconaril or a derivative thereof.

19. An antiviral compound effective against the Ljungan virus of claim 1 for use in the treatment of a disease in a mammal that is caused by infection of the Ljungan virus.

20. The antiviral compound of claim 19, wherein the antiviral compound is Pleconaril or a derivative thereof.
